(19) United States
(12) Patent Application Publication
(10) Pub. No.: US 2026/0000914 A1
(43) Pub. Date: Jan. 1, 2026

(54) MOTION MONITORING USING PROJECTION IMAGING WITH ROTATING VIEWS

(71) Applicants: The University of Sydney, The Univ. of Sydney (AU); Northern Sydney Local Health District, St Leonards (AU)

(72) Inventors: Emily Asuka Hewson, New South Wales (AU); Paul Keall, New South Wales (AU); Owen Thomas Dillon, Sydney (AU); Jeremy Booth, St Leonards (AU)

(73) Assignees: The University of Sydney, The Univ. of Sydney (AU); Northern Sydney Local Health District, St Leonards (AU)

(21) Appl. No.: 18/757,718

(22) Filed: Jun. 28, 2024

(51) Int. Cl. A61N 5/10; G06T 7/246

(52) U.S. Cl. CPC A61N 5/1049; G06T 7/248; A61N 2005/1055; A61N 2005/1062; A61N 5/1078; G06T 2207/30081

(57) ABSTRACT

A method of monitoring motion of an anatomical feature within a subject's body comprises the steps of providing a time series of subject images of a region of the subject's body comprising the anatomical feature, providing a plurality of reference images of the region of the subject's body, determining a displacement of the anatomical feature in each subject image using the plurality of reference mages, and monitoring motion of the anatomical feature based on the displacement of the anatomical feature over the time series of subject images. The plurality of reference images comprise a range of projection angles around an imaging axis and perpendicular to the imaging axis through the region of the subject's body. A computer program product for monitoring motion of an anatomical feature within a subject's body is also provided. A method of delivering therapy to a subject and a therapy delivery system is also provided.

### FIELD

The present disclosure relates to a method of monitoring motion of an anatomical feature within a subject's body, and to a method of delivering therapy to a subject and a therapy delivery system. The present disclosure relates particularly but not exclusively to monitoring motion of the anatomical feature using projection imaging with rotating views.

### BACKGROUND

Treating patients with locally advanced or oligometastatic cancer using radiation therapy typically requires multiple targets to be irradiated simultaneously. However, treatment accuracy may be compromised due to differential motion between independently moving structures^{1,2}. To compensate for relative motion between targets, large planning target volume (PTV) margins can be applied^{3,4} to ensure each target receives the desired prescription dose, however this is a suboptimal solution due to the resulting increase in dose to the healthy tissue⁵.

One patient subset requiring multi-target treatment are those with locally advanced prostate cancer who can benefit from simultaneous prostate and pelvic node irradiation⁶. While the prostate can undergo motion of up to 15 mm⁷, this motion is largely uncorrelated with the pelvic nodes³, which remain approximately fixed to the pelvic vasculature⁸. Thus, when the patient is being aligned for radiation therapy treatment, a compromise must be made when aligning the radiation beam with the targets, and often the prostate is prioritized^{9,10}. Decreases in dose coverage for the lymph nodes of 5%, 15%, and 25% have been seen when a patient is set up with 5 mm, 10 mm, and 15 mm prostate displacements respectively during intensity modulated radiation therapy treatment¹¹.

In addition to translational displacements relative to the prostate, variations in pelvic rotation may also be present, leading to further setup errors. Despite the potential for dosimetric consequences resulting from geometric displacements of the pelvis, the six-degrees-of-freedom (6DoF) position of the pelvis is not monitored and cannot be accounted for during standard radiation therapy treatment without compromising the primary target, e.g., the prostate.

Treatment methods that account for interfraction displacements of the prostate and pelvic lymph nodes through online adaptation have been demonstrated^{12,13}. However, online adaptive methods do not account for patient movement or internal prostate motion during treatment delivery which have shown considerable deviations in some patients^{7,14}.

Existing tumor motion tracking systems such as the CyberKnife¹⁷ and Radixact¹⁸ have been designed to track tumor motion during radiation therapy delivery, however are limited to adapting to single targets. Alternatively, Kilovoltage Intrafraction Monitoring (KIM) uses hardware on a standard linear accelerator to monitor tumor motion in real time by estimating 6DoF motion as observed in intrafraction kV images^{19,20}. KIM has previously been implemented in clinical trials to treat prostate²¹ and liver²² cancer patients, and has demonstrated sub-mm and sub-degree geometric accuracy for monitoring the motion of fiducial markers implanted in the prostate²³. So far, KIM has also been limited to monitoring the motion of a single target to guide motion adaptation.

It would be desirable to provide a method which enables monitoring motion of anatomical features other than the treatment tissue during radiation therapy delivery, particularly tracking of pelvic lymph nodes for treatment of patients with prostate cancer. It would also be desirable to provide a method and system for delivering therapy to a patient based on motion monitoring of multiple targets during radiation therapy delivery, and/or which ameliorates one or more disadvantages of existing arrangements or at least provides a useful alternative.

A reference herein to a patent document or any other matter identified as prior art, is not to be taken as an admission that the document or other matter was known or that the information it contains was part of the common general knowledge as at the priority date of any of the claims.

### Summary of Disclosure

In one aspect, the present disclosure provides a method of monitoring motion of an anatomical feature within a subject's body, the method comprising the steps of: providing a time series of subject images of a region of the subject's body comprising the anatomical feature; providing a plurality of reference images of the region of the subject's body, wherein the plurality of reference images comprise a range of projection angles around an imaging axis and perpendicular to the imaging axis through the region of the subject's body; determining a displacement of the anatomical feature in each subject image using the plurality of reference images; and monitoring motion of the anatomical feature based on the displacement of the anatomical feature over the time series of subject images.

In some embodiments, the step of providing a plurality of reference images comprises providing a plurality of reference images comprising a range of projection angles around a superior-inferior axis through the region of the subject's body, and for each projection angle around the superior-inferior axis, a range of projection angles around the medial-lateral axis through the region of the subject's body.

In some embodiments, the method further comprises the step of processing the plurality of reference images to identify a reference image with a projection angle that corresponds most closely to a location of the anatomical feature in each subject image.

The step of processing the plurality of reference images may comprise calculating a correlation between image values of a first subject image and each of the plurality of reference images, and identifying the reference image with the highest correlation. The step of calculating a correlation may comprise calculating a normalized cross-correlation coefficient between the first subject image and each of the plurality of reference images, and identifying the reference image with the maximum normalized cross-correlation coefficient.

In some embodiments, the step of determining a displacement comprises determining a translation of the anatomical feature along an x-axis (Tₓ) and/or y-axis (T_{y}) based on the respective x and y values that correspond to the maximum normalized cross-correlation coefficient, where the x-axis and y-axis are in a plane perpendicular to a z-axis representing an imaging axis through the region of the subject's body. Upon determining the translation of the anatomical feature for the first subject image, the method may comprise the step of determining the translation of the anatomical feature along the x-axis (Tₓ) and/or y-axis (T_{y}) for subsequent subject images based on translations of the plurality of reference images within a limited range from the first subject image.

In some embodiments, the step of determining a displacement comprises determining a rotation of the anatomical feature around an x-axis (Rₓ), y-axis (R_{y}) and/or z-axis (R_{z}) based on respective x, y and z values that correspond to the maximum normalized cross-correlation coefficient, where the x-axis and y-axis are in a plane perpendicular to the z-axis representing an imaging axis through the region of the subject's body. Upon determining the rotation of the anatomical feature for a first subject image, the method may comprise the step of determining the rotation of the anatomical feature around the x-axis (Rₓ), y-axis (R_{y}) and/or z-axis (R_{z}) for subsequent subject images based on rotations of the plurality of reference images within a limited range from the first subject image.

In some embodiments, the step of determining a displacement comprises determining a translation of the anatomical feature (T_{z}) along a z-axis representing an imaging axis through the region of the subject's body using a probability model based on a location of the anatomical feature in the plurality of reference images. The probability model may be a Gaussian probability density function and the translation of the anatomical feature (T_{z}) may be estimated by the Gaussian distribution along the imaging z-axis.

The step of determining a displacement may comprise determining the displacement of the anatomical feature in six degrees of freedom comprising translation and rotation (Tₓ, T_{y}, T_{z}, Rₓ, R_{y}, R_{z}). The method may further comprise the step of transforming the displacement of the anatomical feature (Tₓ, T_{y}, T_{z}, Rₓ, R_{y}, R_{z}) to a frame of reference of the subject's body (T_{LR}, T_{SI}, T_{AP}, R_{LR}, R_{SI}, R_{AP}).

In some embodiments, one or more steps of the method are performed during delivery of therapy to the subject for monitoring in vivo motion of the anatomical feature within the subject's body. The step of providing the time series of subject images may be performed during delivery of therapy to the subject. In some embodiments, all steps of the method are performed during delivery of therapy to the subject. The method may also be performed in real time for monitoring in vivo motion of the anatomical feature within the subject's body.

In some embodiments, the anatomical feature is a bony structure of the subject. The anatomical feature may be the pelvis of the subject.

In another aspect, the present disclosure provides a computer program product for monitoring motion of an anatomical feature within a subject's body, the computer program product comprising a non-transitory computer readable storage medium having program code embodied therewith, the program code executable by one or more processors, to perform a method comprising the steps of: providing a time series of subject images of a region of the subject's body comprising the anatomical feature; providing a plurality of reference images of the region of the subject's body, wherein the plurality of reference images comprise a range of projection angles around an imaging axis and perpendicular to the imaging axis through the region of the subject's body; determining a displacement of the anatomical feature in each subject image using the plurality of reference images; and monitoring motion of the anatomical feature based on the displacement of the anatomical feature over the time series of subject images.

In some embodiments, the step of providing a plurality of reference images comprises providing a plurality of reference images comprising a range of projection angles around a superior-inferior axis through the region of the subject's body, and for each projection angle around the superior-inferior axis, a range of projection angles around the medial-lateral axis through the region of the subject's body.

In some embodiments, the method further comprises the step of processing the plurality of reference images to identify a reference image with a projection angle that corresponds most closely to a location of the anatomical feature in each subject image.

The step of processing the plurality of reference images may comprise calculating a correlation between image values of a first subject image and each of the plurality of reference images, and identifying the reference image with the highest correlation. The step of calculating a correlation may comprise calculating a normalized cross-correlation coefficient between the first subject image and each of the plurality of reference images, and identifying the reference image with the maximum normalized cross-correlation coefficient.

In some embodiments, the step of determining a displacement comprises determining a translation of the anatomical feature along an x-axis (Tₓ) and/or y-axis (T_{y}) based on the respective x and y values that correspond to the maximum normalized cross-correlation coefficient, where the x-axis and y-axis are in a plane perpendicular to a z-axis representing an imaging axis through the region of the subject's body. Upon determining the translation of the anatomical feature for the first subject image, the method may comprise the step of determining the translation of the anatomical feature along the x-axis (Tₓ) and/or y-axis (T_{y}) for subsequent subject images based on translations of the plurality of reference images within a limited range from the first subject image.

In some embodiments, the step of determining a displacement comprises determining a rotation of the anatomical feature around an x-axis (Rₓ), y-axis (R_{y}) and/or z-axis (R_{z}) based on respective x, y and z values that correspond to the maximum normalized cross-correlation coefficient, where the x-axis and y-axis are in a plane perpendicular to the z-axis representing an imaging axis through the region of the subject's body. Upon determining the rotation of the anatomical feature for a first subject image, the method may comprise the step of determining the rotation of the anatomical feature around the x-axis (Rₓ), y-axis (R_{y}) and/or z-axis (R_{z}) for subsequent subject images based on rotations of the plurality of reference images within a limited range from the first subject image.

In some embodiments, the step of determining a displacement comprises determining a translation of the anatomical feature (T_{z}) along a z-axis representing an imaging axis through the region of the subject's body using a probability model based on a location of the anatomical feature in the plurality of reference images. The probability model may be a Gaussian probability density function and the translation of the anatomical feature (T_{z}) may be estimated by the Gaussian distribution along the imaging z-axis.

The step of determining a displacement may comprise determining the displacement of the anatomical feature in six degrees of freedom comprising translation and rotation (Tₓ, T_{y}, T_{z}, Rₓ, R_{y}, R_{z}). The method may further comprise the step of transforming the displacement of the anatomical feature (Tₓ, T_{y}, T_{z}, Rₓ, R_{y}, R_{z}) to a frame of reference of the subject's body (T_{LR}, T_{SI}, T_{AP}, R_{LR}, R_{SI}, R_{AP}).

In some embodiments, one or more steps of the method are performed during delivery of therapy to the subject for monitoring in vivo motion of the anatomical feature within the subject's body. The step of providing the time series of subject images may be performed during delivery of therapy to the subject. In some embodiments, all steps of the method are performed during delivery of therapy to the subject. The method may also be performed in real time for monitoring in vivo motion of the anatomical feature within the subject's body.

In some embodiments, the anatomical feature is a bony structure of the subject. The anatomical feature may be the pelvis of the subject.

In another aspect, the present disclosure provides a method of delivering therapy to a subject, the method comprising the steps of: providing motion data of a first anatomical feature within a region of the subject's body, the motion data being determined according to a method of monitoring motion of an anatomical feature within a subject's body of any one of the embodiments of the above aspect; determining one or more target locations within the region of the subject's body for delivering therapy based on the provided motion data of the first anatomical feature; and operating a therapy delivery system to deliver therapy to the subject at the one or more target locations.

In some embodiments, the method further comprises the steps of providing motion data of a second anatomical feature within the region of the subject's body, and determining one or more target locations within the region of the subject's body for delivering therapy based on the provided motion data of the first anatomical feature and the second anatomical feature.

In some embodiments, the step of determining one or more target locations comprises determining a target location for delivering therapy to each of the first anatomical feature and the second anatomical feature based on the provided motion data of the first anatomical feature and the second anatomical feature, and operating the therapy delivery system to simultaneously deliver therapy to the subject at both target locations of the first anatomical feature and the second anatomical feature.

In some embodiments, one or more steps of the method are performed during delivery of therapy to the subject. The step of providing motion data of the first anatomical feature and/or the second anatomical feature may be performed during delivery of therapy to the subject for monitoring in vivo motion of the first anatomical feature and/or the second anatomical feature within the subject's body. The step of determining one or more target locations may also be performed during delivery of therapy to the subject. The steps of providing motion data and determining one or more target locations may also be performed in real time.

In some embodiments, the first anatomical feature is the pelvis of the subject, and the second anatomical feature is the prostate of the subject.

In another aspect, the present disclosure provides a method of delivering therapy to a subject, the method comprising the steps of: providing motion data of a first anatomical feature within a region of the subject's body, the motion data being determined according to a method of monitoring motion of an anatomical feature within a subject's body, the method of monitoring motion comprising the steps of: providing a time series of subject images of a region of the subject's body comprising the anatomical feature; providing a plurality of reference images through the region of the subject's body, wherein the plurality of reference images comprise a range of projection angles around an imaging axis and perpendicular to the imaging axis through the region of the subject's body; determining a displacement of the anatomical feature in each subject image using the plurality of reference images; and monitoring motion of the anatomical feature based on the displacement of the anatomical feature over the time series of subject images; determining one or more target locations within the region of the subject's body for delivering therapy based on the provided motion data of the first anatomical feature; and operating a therapy delivery system to deliver therapy to the subject at the one or more target locations.

In another aspect, the present disclosure provides a therapy delivery system comprising: a therapy system for delivering therapy to a subject; an imaging system for imaging a region of the subject's body; one or more processors configured to: provide motion data of a first anatomical feature within the region of the subject's body, the motion data being determined according to a method of monitoring motion of an anatomical feature within a subject's body of any one of the embodiments of the above aspect; and determine one or more target locations within the region of the subject's body for delivering therapy based on the provided motion data of the first anatomical feature; and a controller configured to operate the therapy delivery system to deliver therapy to the subject at the one or more target locations.

In some embodiments, the one or more processors are further configured to provide motion data of a second anatomical feature within the region of the subject's body, and determine one or more target locations within the region of the subject's body for delivering therapy based on the provided motion data of the first anatomical feature and the second anatomical feature.

In some embodiments, the one or more processors are configured to determine a target location for delivering therapy to each of the first anatomical feature and the second anatomical feature based on the provided motion data of the first anatomical feature and the second anatomical feature. The controller may be configured to operate the therapy delivery system to simultaneously deliver therapy to the subject at both target locations of the first anatomical feature and the second anatomical feature.

In some embodiments, the one or more processors are configured to provide motion data of the first anatomical feature and/or the second anatomical feature during delivery of therapy to the subject for monitoring in vivo motion of the first anatomical feature and/or the second anatomical feature within the subject's body. The one or more processors may also be configured to determine one or more target locations during delivery of therapy to the subject. The one or more processors may also be configured to provide motion data and determining one or more target locations in real time.

In some embodiments, the first anatomical feature is the pelvis of the subject, and the second anatomical feature is the prostate of the subject.

In another aspect, the present disclosure provides a therapy delivery system comprising: a therapy system for delivering therapy to a subject; an imaging system for imaging a region of the subject's body; one or more processors configured to: provide motion data of a first anatomical feature within the region of the subject's body, the motion data being determined according to a method of monitoring motion of an anatomical feature within a subject's body, the method of monitoring motion comprising the steps of: providing a time series of subject images of a region of the subject's body comprising the anatomical feature; providing a plurality of reference images through the region of the subject's body, wherein the plurality of reference images comprise a range of projection angles around an imaging axis and perpendicular to the imaging axis through the region of the subject's body; determining a displacement of the anatomical feature in each subject image using the plurality of reference images; and monitoring motion of the anatomical feature based on the displacement of the anatomical feature over the time series of subject images; and determine one or more target locations within the region of the subject's body for delivering therapy based on the provided motion data of the first anatomical feature; and a controller configured to operate the therapy delivery system to deliver therapy to the subject at the one or more target locations.

### BRIEF DESCRIPTION OF DRAWINGS

The present disclosure will now be described in greater detail with reference to the accompanying drawings in which like features are represented by like numerals. It is to be understood that the embodiments shown are examples only and are not to be taken as limiting the scope of the present disclosure as defined in the claims appended hereto.

FIG. 1 is a perspective diagram of an existing therapy delivery system including a linear accelerator for delivering radiation therapy to a subject and an on-board imaging system for acquiring one or more images of the subject during therapy, showing an imaging beam and projection angle in broken lines.

FIG. 2 is another perspective diagram of the existing therapy delivery system according to FIG. 1, with the subject not present and the linear accelerator being rotated 90 degrees anticlockwise to illustrate the imaging beam and projection line in broken lines now directed towards a floor.

FIG. 3 is a flow chart illustrating steps in a method of monitoring motion of an anatomical feature within a subject's body, with optional steps shown in broken lines, according to some embodiments of the disclosure.

FIG. 4 is a schematic diagram illustrating additional steps in the method of FIG. 3, including generating a plurality of reference images before the subject undergoes therapy, where the anatomical feature is the pelvis, with optional steps shown in broken lines, according to some embodiments of the disclosure.

FIG. 5 is a schematic diagram illustrating additional steps in the method of FIGS. 3 and 4, including determining displacement of the pelvis using images of the subject acquired during therapy, with optional steps shown in broken lines, according to some embodiments of the disclosure.

FIG. 6 is a schematic diagram illustrating additional steps in the method of FIG. 5 relating to template matching for identifying a reference image with a maximum normalized cross-correlation for a range of angles to determine displacement of the pelvis, according to some embodiments of the disclosure.

FIG. 7 is schematic diagram illustrating a method of monitoring motion of another anatomical feature of the subject, in this case the prostate, and outputting the prostate displacement in six degrees of freedom (DoF), with optional steps shown in broken lines, according to some embodiments of the disclosure.

FIG. 8 is another flow chart illustrating a method of delivering therapy to a subject, including the steps of providing motion data of a first anatomical feature (e.g., the pelvis) and optionally, a second anatomical feature (e.g., the prostate) of the subject as shown in broken lines, and determining one or more target locations for delivering therapy, according to some embodiments of the disclosure.

FIG. 9 is another flow chart illustrating a method of delivering therapy to a subject, including the steps from the method of FIGS. 3 to 7 and determining target location(s) for delivering therapy, with optional post-treatment evaluation of accuracy of the method shown in broken lines, according to some embodiments of the disclosure.

FIG. 10 is a schematic diagram illustrating additional steps in the method of FIG. 9 relating to post-treatment evaluation of accuracy of the method, according to some embodiments of the disclosure.

FIG. 11 is a schematic diagram illustrating a therapy delivery system which includes either an on-board or off-board computer system with a processing unit to determine target location(s) for delivering therapy, and a controller to operate the therapy system to delivery therapy to the subject, according to some embodiments of the disclosure.

FIGS. 12 and 13 are charts illustrating Example 1 which evaluates the motion monitoring method for translation (FIG. 12) and rotation (FIG. 13) in the patient coordinate system for 20 patients across five fractions, illustrating the geometric error where the whiskers represent the minimum and maximum values of the error, according to some embodiments of the disclosure.

FIGS. 14 to 19 are charts illustrating the distribution of relative displacements between the pelvic bone and prostate observed in the kV projections, namely in histograms of the relative translational (FIGS. 14, 16 and 18) and rotational (FIGS. 15, 17 and 19) displacements observed between the pelvic bone and prostate across all kV images for the 20 patients analyzed in Example 1, according to some embodiments of the disclosure.

FIGS. 20 and 21 are charts illustrating the relative motion observed for one patient, where the translation (FIG. 20) and rotation (FIG. 21) is observed for the pelvic bone (solid lines) and prostate (broken lines) in Example 1, according to some embodiments of the disclosure.

FIGS. 22 and 23 are charts illustrating the resulting interfraction displacements of the pelvic bone for translation (FIG. 22) and rotation (FIG. 23) after patient setup for each patient across five fractions by applying a translational shift to correctly align the prostate to the isocenter for the beginning of treatment in Example 1, according to some embodiments of the disclosure.

### DESCRIPTION OF EMBODIMENTS

Embodiments of the present disclosure are discussed herein by reference to the drawings which are not to scale and are intended merely to assist with explanation of the present disclosure.

Reference herein to a subject may include a human or animal subject, or a human or animal patient on which medical procedures are performed and/or screening, monitoring and/or diagnosis of a disease or disorder is performed. In relation to animal patients, embodiments of the disclosure may also be suitable for veterinary applications. The terms “subject” and “patient” are used interchangeably throughout the specification and should be understood to represent the same feature of embodiments of the disclosure.

Reference herein is also provided to anatomical planes of a subject's body and anatomical axes through a subject's body. The anatomical planes include a traverse, sagittal and coronal plane of the subject's body. The anatomical axes include a medial-lateral (RL) axis, a superior-inferior (SI) axis and an anterior-posterior (AP) axis through the subject's body. In most embodiments of the disclosure, the subject's body is in a supine position as the subject is positioned lying horizontally on a couch or tray of a therapy delivery system. The transverse plane includes a right-to-left or RL axis passing horizontally through the subject's body in the supine position. The sagittal plane includes a head-to-toe or SI axis passing horizontally through the subject's body in the supine position. The coronal plane includes a front-to-back or AP axis passing vertically through the subject's body in the supine position.

Reference herein is also provided to therapy of a subject. The therapy may include delivering radiation therapy to the subject, e.g., electromagnetic radiation to treat a cancerous tumor within the subject's body. The electromagnetic radiation may include high-energy particles or waves such as x-rays, gamma rays, electron beams, or protons, to destroy or damage cancer cells. However, embodiments of the disclosure are not limited to radiation therapy and may encompass other forms of therapy in which energy is delivered to the subject for treatment of a disease or disorder, such as ultrasound, vibration, heat, or light energy.

Reference herein is also provided to treatment of a subject. Treatment may refer to treating a disease or disorder of the subject's body, e.g., cancer and particularly in this specification, prostate cancer, or other forms of cancer such as lung and oligometastatic cancer. However, embodiments of the disclosure are not limited to cancer treatment and may include treatment of other diseases or disorders of the subject's body. The treatment may include the delivery of therapy to the subject, e.g., radiation therapy or otherwise, as discussed above.

Embodiments of the disclosure are directed to a method of monitoring motion of an anatomical feature within a region of a subject's body. The method may monitor in vivo motion of the anatomical feature within the subject's body. The method may be performed in real time. The method may be performed during delivery of therapy to the subject.

The region of the subject's body may include the abdomen, such as the lower or upper abdomen, and more particularly, may include the pelvic region or spinal region of the subject's body. The anatomical feature may include an anatomical structure within a region of the subject's body. The anatomical structure may include a bony structure of the subject's body such as the pelvis or one or more vertebrae. The anatomical feature may include one or more bones or bone portions of the subject's body. In other embodiments, the anatomical feature may include non-bony structures such as tissues, organs, glands or membranes within the subject's body. For example, the anatomical feature may include one or more vesicles, such as the seminal vesicles within the pelvic region of the subject's body, or one or more nearby organs located within the region of the subject's body.

Embodiments of the disclosure have particularly utility in monitoring motion of a bony structure within the subject's body, and in particular, the pelvis or one or more pelvic bones. The pelvis acts as a surrogate for the location of the pelvic lymph nodes in the subject's body. It is desirable to provide multi-targeted treatment for patients with locally advanced prostate cancer through simultaneous irradiation of the prostate and the pelvic lymph nodes. However, the prostate and pelvis can both undergo motion within the subject's body or through movement of the patient before or during treatment such that the radiation beam is not correctly aligned with the targets.

Embodiments of the present disclosure may enable multi-target motion monitoring by providing a method to monitor motion of the pelvis by calculating the translation and rotation of the pelvic bone in intrafraction kV images as a surrogate for pelvic lymph node displacement. The resulting method is integrated into a method and system of delivering therapy to a subject which in some embodiments enables simultaneous real-time motion monitoring of both the prostate and pelvic lymph nodes through the pelvis acting as a surrogate, and multi-targeted treatment adaptation and delivery of therapy.

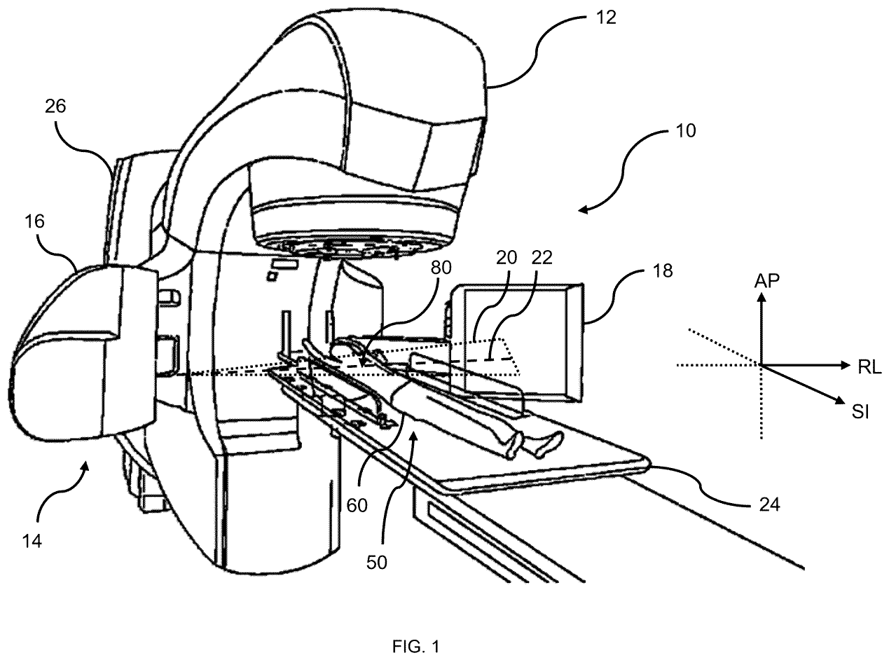

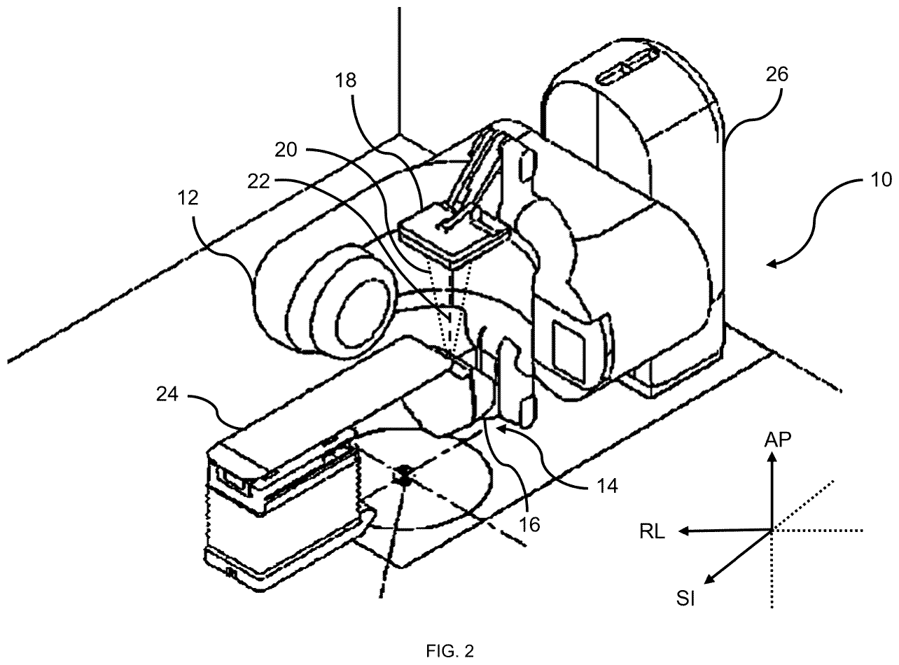

By way of background, FIGS. 1 and 2 are provided which are perspective diagrams showing the features of an existing therapy delivery system 10. The exemplary therapy delivery system 10 is a TrueBeam radiotherapy system by Varian Medical Systems, Inc. designed for radiation therapy delivery for cancer treatment. However, it is to be appreciated that in various embodiments of the disclosure other therapy delivery systems 10 may be employed, such as ultrasound therapy systems.

The therapy delivery system 10 includes a linear accelerator 12 supported by a gantry 26 for delivering radiation therapy to a subject 50. The subject 50 is positioned on a couch or tray 24 of the therapy delivery system 10 in a supine position. An on-board imaging system 14 is provided for acquiring one or more images of the subject 50 during treatment to monitor position of the tumor or structural features to be irradiated. The imaging system 14 includes a source 16 for generating energy, such as x-ray radiation, and a detector 18 for detection of x-rays to acquire the images.

FIG. 1 illustrates an imaging beam 20 generated by the imaging system 14 with a projection line 22 as shown in broken lines. In FIG. 2, the subject has been removed for clarity and the linear accelerator 12 has been rotated 90 degrees anticlockwise by adjusting the angle of the gantry 26. The imaging beam 20 with projection line 22 is now directed towards a floor of the room in which the therapy delivery system 10 is located.

FIGS. 1 and 2 also illustrate a three-dimensional frame of reference for the subject 50. The subject 50 is in a supine or horizontal upright position on the tray 24. The three axes for the subject's frame of reference include the superior-inferior (SI) axis, the medial-lateral or right-left (RL) axis, and the anterior-posterior (AP) axis through the subject's body.

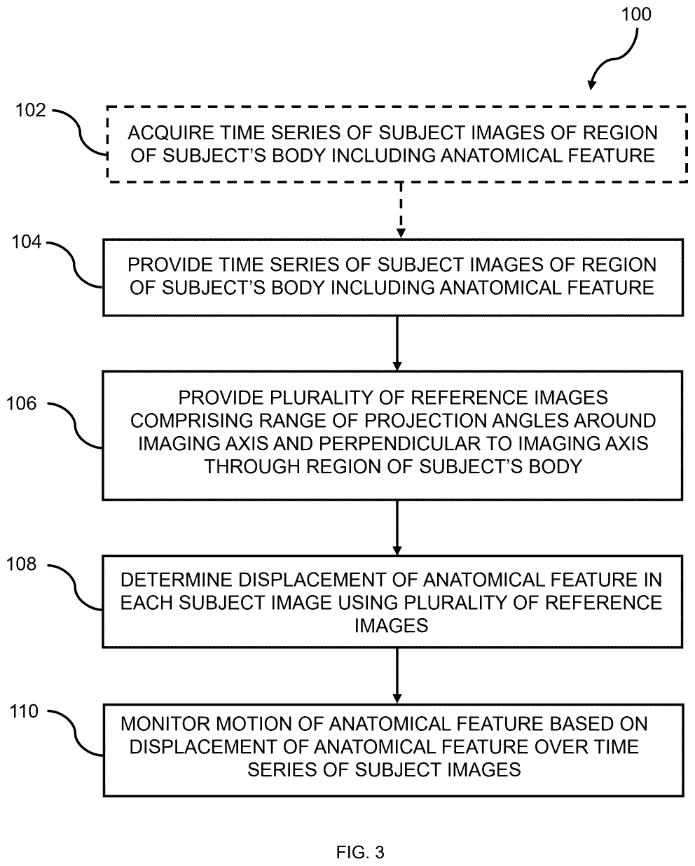

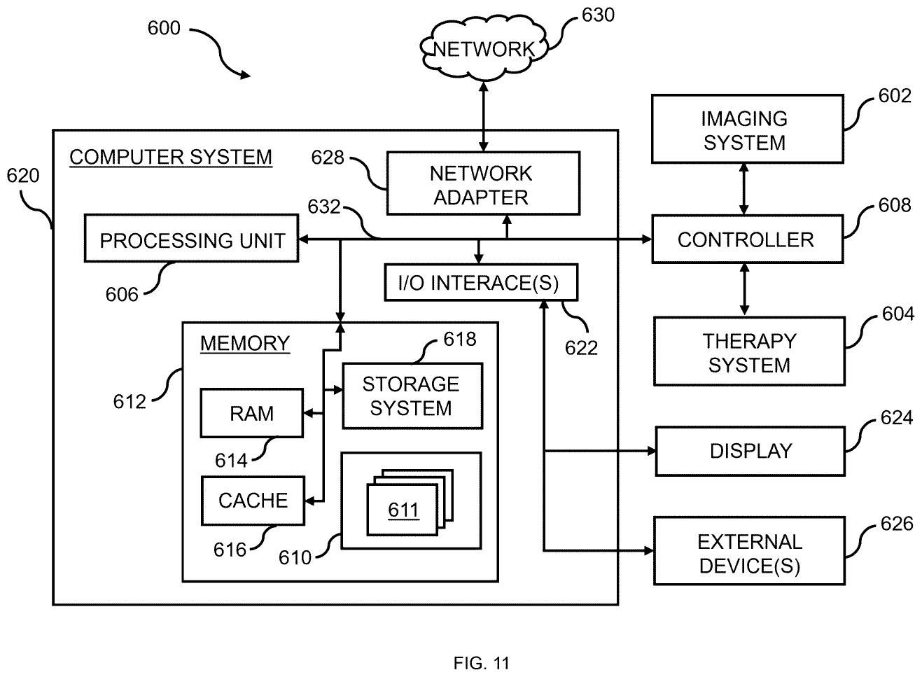

FIG. 3 is a flow chart illustrating steps in a method 100 of monitoring motion of an anatomical feature 70 within a subject's body 60 (see FIG. 1), with optional steps shown in broken lines, according to some embodiments of the disclosure. The method 100 comprises an optional step 102 of acquiring a time series of subject images 30 of a region 80 of a subject's body 60 including the anatomical feature 70. The time series of subject images 30 may be acquired using an existing therapy delivery system 10 as described with respect to FIGS. 1 and 2, or a novel therapy delivery system 600 as will be described with respect to FIG. 11. However, the method 100 does not require acquisition of the subject images 30, which may have already occurred and images be provided ready for processing.

The method 100 begins with step 102 of providing a time series of subject images 30 of a region 80 of the subject's body 60 comprising the anatomical feature 70. The time series of subject images 30 may include a sequence of subject images 30 that are acquired at a predetermined frequency over a period of time. The time series of subject images 30 may be intrafraction kV images that are acquired between fractions, i.e., periods of time during which therapy is delivered to the subject 50 by the therapy delivery system 10, 600. The step 102 of providing the time series of subject images 30 may include receiving the time series of subject images 30 from an imaging system 16, 602 of a therapy delivery system 10, 600 (see FIGS. 1, 2 and 11). The time series of subject images 30 may be received at a processing unit 606 comprising one or more processors of the therapy delivery system 600 (see FIG. 11).

The method 100 then includes the step 106 of providing a plurality of reference images 40 of the region 80 of the subject's body 60. The plurality of reference images 40 comprise a range of projection angles around an imaging axis (z-axis) and perpendicular to the imaging axis (z-axis) through the region 80 of the subject's body 60. The method 100 then includes the step 108 of determining a displacement of the anatomical feature 70 in each subject image 30 using the plurality of reference images 40. Finally, the method 100 concludes with the step 110 of monitoring motion of the anatomical feature 70 based on the displacement of the anatomical feature 70 over the time series of subject images 30. The motion may be monitored based on differences in the determined displacement values of the anatomical feature 70 over the time series of subject images 30.

The step 106 of providing the plurality of reference images 40 may include receiving the plurality of reference images 40 from a memory 612 or via a network 630 of a therapy delivery system 600 (see FIG. 11). The plurality of reference images 40 may be stored on-board or off-board, such as in the storage system 618 of memory 612 or accessible via the network 630 such as via a Cloud storage system (see FIG. 11). The plurality of reference images 40 may be received at a processing unit 606 comprising one or more processors of the therapy delivery system 600 (see FIG. 11). The steps 108, 110 of the method 100 may be performed by one or more processors, e.g., of the processing unit 606 of the therapy delivery system 600 (see FIG. 11), for determining displacement of the anatomical feature 70 in each subject image 30 using the plurality of reference images 40 and monitoring motion of the anatomical feature 70 based on the displacement of the anatomical feature 70 over the time series of subject images 30.

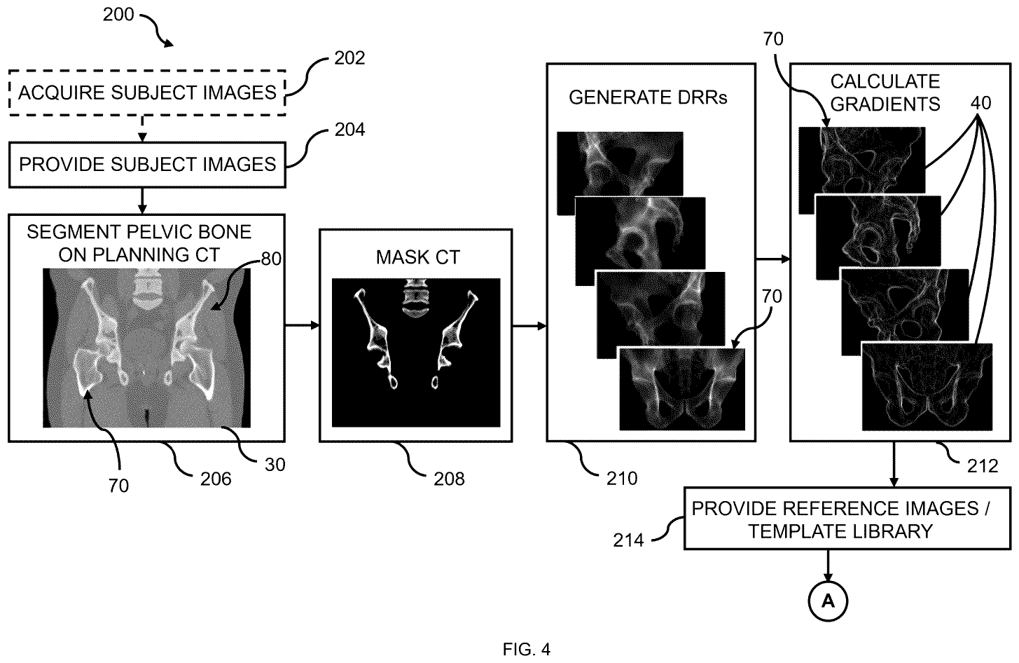

FIG. 4 is a schematic diagram illustrating additional steps in the method 100 of FIG. 3, including a method 200 of generating a plurality of reference images 40 prior to treatment, where the anatomical feature 70 is the pelvis, with optional steps shown in broken lines, according to some embodiments of the disclosure. FIG. 4 includes an optional step 202 of acquiring a plurality of subject images 30 (e.g., using an imaging system 16, 602 of a therapy delivery system 10, 600 or another imaging system such as a CT imaging system). However, the method 200 does not require acquisition of the subject images 30, which may have already occurred and be ready for processing. The first step 204 is to provide a plurality of subject images 30 which are ready for processing. The subject images 30 may be a time series of images. The time series of subject images 30 may include a sequence of subject images 30 that are acquired at a predetermined frequency over a period of time.

In a first step 206, the pelvic bone 70 is segmented on the planning CT image of the region 80 of the subject's body 60. The pelvic bone 70 on each patient's planning CT is contoured and used to mask the CT volume in step 208. The femurs are excluded from the contour. The segmentation and masking steps 206, 208 may be performed by various software tools including 3D Slicer, MATLAB functions or treatment planning software such as Eclipse, which are available in imaging processing of CT images. Embodiments of the disclosure are not limited to these software tools as would be appreciated by a person skilled in the art. The method 200 then involves the step 210 of generating digitally reconstructed radiographs (DRRs) of the masked CT volumes. This step may be performed by various software tools available in image processing of CT images, such as by using the open-source Reconstruction Toolkit (RTK)²⁵. Since the shape of the pelvic bone 70 on the 2D treatment images varies depending on the gantry angle, it is desirable to generate DRRs at a range of projection angles to form a library with six degrees of freedom (6DoF) for each patient 50 at step 210.

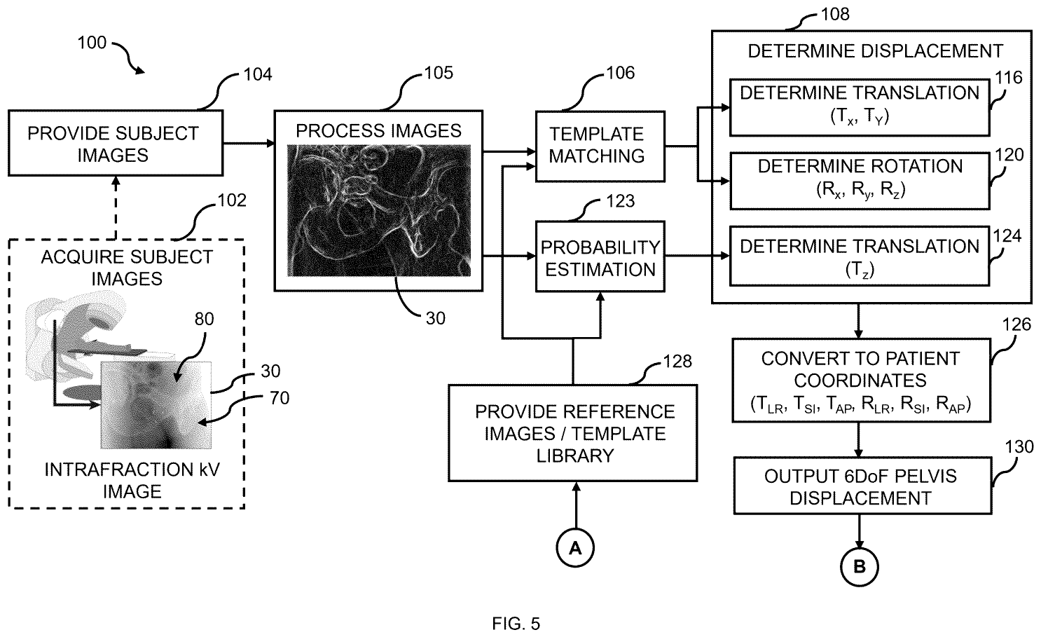

The subject images 30 may thus be acquired at step 202 or provided at step 204 with a range of projection angles. A plurality of images having projection angles around the superior-inferior (SI) axis may be provided (see FIGS. 1 and 2). For example, projection angles from 0° to 359.5° may be generated, with intervals of 0.5°. Furthermore, for each projection SI angle, projections with the pelvic bone 70 rotating out of the imaging plane may be also generated (x-axis, see FIG. 5). For example, projection angles from −6° to +6° degrees may be generated, with intervals of 0.5°. It is to be appreciated that embodiments of the disclosure are not limited to the ranges of projection angles and intervals described herein. Additional projection angles with a smaller interval could be generated or acquired to provide a more extensive library of template or reference images, although optimisation is necessary to avoid significant computation time and delays in steps of the method 100.

A plurality of images having projection angles from 0° to 360° could be generated around the SI axis, and for each projection SI angle, a plurality of images having projection angles from −180° to +180° could be generated around the medial-lateral (RL) axis. In one embodiment, a plurality of images may be generated with projection angles from 90° to 270° with intervals of 1.0° around the SI axis, and for each projection SI angle, a plurality of images may be generated from −10° to +10° degrees with 1.0° degree intervals around the RL axis. The range of projection angles and intervals may depend on the anatomical feature to be monitored and/or the therapy or treatment being delivered to the subject 50. For example, large ranges of projection angles could be used if large rotations during treatment are expected, or the range could be limited around the SI axis if the treatment is only delivered at certain angles around the subject 50 and the full 360° is not required (e.g., depending on the treatment and therapy delivery system).

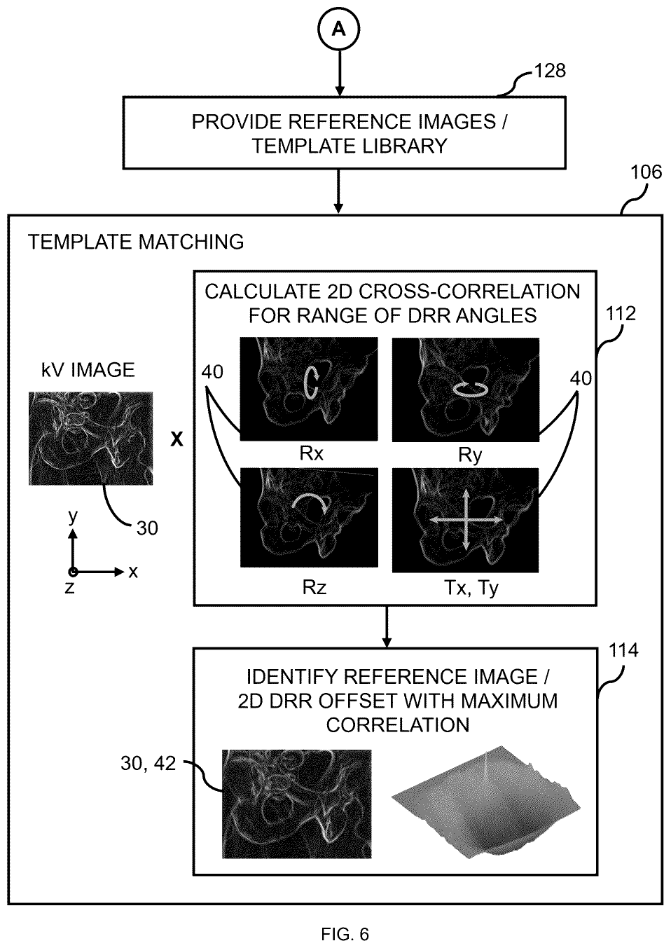

After the DRRs are generated at step 210, the method 200 further includes the step 212 of calculating the magnitude of the gradients for each DRR in the x and y directions on the 2D images so that the edges of the bony anatomy form the dominant feature in each reference image 40. This step may be performed by various software tools available to a person skilled in the art, including MATLAB functions and programming of the calculations in MATLAB or Python, for example. The template generation method 200 may finally include the step 214 of providing a plurality of reference images 40 or a template library which is used in embodiments of the method 100 for monitoring motion of an anatomical feature 70 within a subject's body 60. The reference images 40 comprise a range of projection angles around the superior-inferior (SI) axis through the region 80 of the subject's body 60, and for each projection angle around the SI axis, a range of projection angles may be provided around the medial-lateral or right-left (RL) axis through the region 80 of the subject's body 60. The step 214 of providing the reference images 40 may include outputting the reference images for use in the method 100 of the embodiments shown in FIGS. 5 and 6 as indicated by the arrow with the encircled letter “A”. The reference images 40 may be stored in a memory 612 in a storage system 618 on-board a therapy delivery system 600 or accessible from a network 630 via a network adapter 628 (see FIG. 11) for processing by a processing unit 606 in the method 100.

FIG. 5 is a schematic diagram illustrating additional steps in the method 100 of FIG. 3, including a step 108 of determining displacement of the pelvis 70 using images of the subject 30 acquired during therapy, with optional steps shown in broken lines, according to some embodiments of the disclosure.

As discussed above in relation to the method 100 of FIG. 3, the method may optionally include the step 102 of acquiring subject images 30, although these may have already been acquired. The method 100 includes the step 104 of providing a time series of subject images 30 of the region 80 of the subject's body 60. The processing step 105 includes processing of the subject images 30 before the step of determining displacement 108 of the pelvis 70. The processing step 105 may be performed by one or more processors of a processing unit 606 of a therapy delivery system 600 as shown in FIG. 11. The processing may include implementing a method of three-frame temporal averaging, and applying a median filter to reduce image noise, following by a Gaussian bandpass filter to enhance the edges of the bony anatomy in the image 30. Any fiducial markers implanted in the region 80 of the subject's body 60 (e.g., in the prostate for tracking of this organ) may be automatically masked in the kV images 30. The masking may be achieved by various imaging processing techniques including replacement of the marker in the images with background noise. The gradients of each kV image 30 may be calculated consistent with the step 210 of the template generation method 200 of FIG. 4 to generate an image that highlights the edges of the bony anatomy.

The method 100 may further include a template matching step 106 which will be described in more detail with respect to FIG. 6. The template matching step 106 may be performed by one or more processors of a processing unit 606 of a therapy delivery system 600 as shown in FIG. 11. The template matching step 106 is required to determine translation and rotation in all degrees of freedom except for the translation in the imaging z-axis (T_{z}). For the translation in the imaging z-axis (T_{z}), a probability estimation step 123 is required. Both the template matching step 106 and probability estimation step 123 require use of the plurality of reference images 40 or template library that were prepared in the method 200 of FIG. 4 as indicated by the arrow with the encircled letter “A” in FIG. 5.

FIG. 6 is a schematic diagram illustrating additional steps in the method 100 of FIG. 5 relating to template matching 106 for processing the plurality of reference images 40 to identify a reference image 42 with a projection angle that corresponds most closely to a location of the anatomical feature 70, i.e., the pelvis, in each subject image 30. The method 100 includes the step 128 of providing the plurality of reference images 40 or the template library that were prepared in the method 200 of FIG. 4 as indicated by the arrow with the encircled letter “A”. The template matching step 106 includes a step 112 of calculating a correlation between image values of a first subject image 30 and each of the plurality of reference images 40, and a step 114 of identifying the reference image 42 with the highest correlation. In particular, a two-dimensional (2D) normalized cross-correlation coefficient between the first subject image 30 and each of the plurality of reference images may be calculated at step 112, and the reference image 42 identified at step 114 with the maximum normalized cross-correlation coefficient. It is to be noted that the image shown at step 114 is a kV image of the subject image 30 with the identified reference image 42 overlayed.

In step 112, the kV image 30 is compared with a range of angles of the digitally reconstructed radiographs (DRRs) of the reference images 40. A translation of the pelvis 70 along an x-axis (Tₓ) and y-axis (T_{y}) is determined at step 116 of FIG. 5 based on the respective x and y values that correspond to the maximum normalized cross-correlation coefficient of the reference image 42. The x-axis and y-axis are in a plane perpendicular to a z-axis representing an imaging axis through the region 80 of the subject's body 60. The axes are shown below the kV image 30 in the template matching step 106 of FIG. 5, where the x-axis and y-axis form the respective horizontal and vertical axes of the kV image 30 and the z-axis is into the page.

After translation of the anatomical feature 70 has been determined for a first subject image 30, the method 100 may comprise determining the translation of the anatomical feature or pelvis 70 along the x-axis (Tₓ) and/or y-axis (T_{y}) for subsequent subject images based on translations of the plurality of reference images 40 within a limited range from the first subject image 30. For example, the limited range may be within 1 mm in each direction from the first subject image.

The template matching method 106 may also include repeating the calculation of the normalized cross-correlation coefficient for a selection of DRRs from the generated template library 40 to check for rotations close to the expected rotation of the anatomical feature or pelvis 70 in the Rₓ and R_{y} directions. A rotation of the pelvis 70 around an x-axis (Rₓ), y-axis (R_{y}) and/or z-axis (R_{z}) is determined at step 120 of FIG. 5 based on respective x, y and z values that correspond to the maximum normalized cross-correlation coefficient of the reference image 42. The rotation may be determined by rotating each template image 40 around the imaging z-axis to calculate the normalized cross-correlation coefficient for varying R_{z} rotations. The DRR 42 and R_{z} rotation with the highest normalized cross-correlation value may be used to determine the magnitude of Rₓ, R_{y} and R_{z}. For the first treatment image 30, rotations ranging ±6° from the planned pelvic bone position were calculated. It is to be appreciated that rotations in other ranges for a first subject image may be used in embodiments of the disclosure, including rotations in the range of ±10° or ±20°, for example, or even in the range of ±180° although this is unlikely since such rotations would not be realistic. Similarly, after rotation of the anatomical feature 70 has been determined for a first subject image 30, the method 100 may comprise determining the rotation of the anatomical feature around the x-axis (Rₓ), y-axis (R_{y}) and/or z-axis (R_{z}) for subsequent subject images based on rotations of the plurality of reference images 40 within a limited range from the first subject image 30. For example, the limited range may be within +0.5° from the first subject image 30. It is to be appreciated that rotations in other ranges for subsequent subject images may be used in embodiments of the disclosure, including rotations in the range of +1.5° or +5°, for example, or even in the range of +180° although this is unlikely since such rotations would not be realistic.

Returning to FIG. 5, the step 108 of determining displacement of the anatomical feature 70 may further comprise the step 124 of determining a translation of the anatomical feature (T_{z}) along a z-axis representing an imaging axis through the region 80 of the subject's body 60 (see also FIG. 6). The translation along the z-axis (T_{z}) may be calculated using a probability model determined at step 123 which is based on a location of the anatomical structure 70 in the plurality of reference images 40 provided at step 128 from generation in the method 200 of FIG. 4. The probability model may be a method previously described by Poulson et al²⁶. The probability model may be a Gaussian probability density function (PDF) and the translation of the anatomical feature (T_{z}) is estimated by the Gaussian distribution along the imaging z-axis. An initial PDF may be built using the position of the pelvis observed in a number of projections acquired using maximum likelihood estimation. Then, the unknown position of the pelvis along the direction of the imager axis may be estimated by finding the expectation value determined by the 1D Gaussian distribution along the imaging z-axis.

Preferably, the method 100 includes determining displacement of the anatomical feature 70, e.g., the pelvis, in six degrees of freedom (6DoF) comprising translation (Tₓ, T_{y}, T_{z}) and rotation (Rₓ, R_{y}, R_{z}) of the anatomical feature 70. The method 100 may further include the step 126 shown in FIG. 5 of converting the displacement to the patient coordinates. The step 126 may include applying a rotational transformation to determine the pelvic bone displacement in the present example in the patient coordinates with respect to the planning CT. The displacement of the anatomical feature (Tₓ, T_{y}, T_{z}, Rₓ, R_{y}, R_{z}) may be transformed to a frame of reference of the subject's body (T_{LR}, T_{SI}, T_{AP}, R_{LR}, R_{SI}, R_{AP}). The determining displacement step 108 as described above, including one or more of the steps 116, 120 and 124 of determining translation and rotation, and the step 126 of converting the displacement to the patient coordinates, may be performed by one or more processors of a processing unit 606 of a therapy delivery system 600 as shown in FIG. 11.

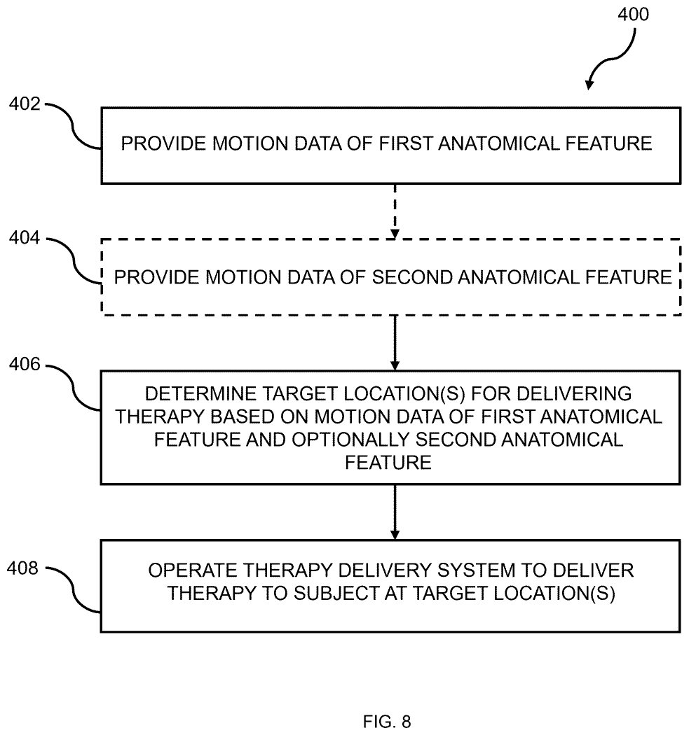

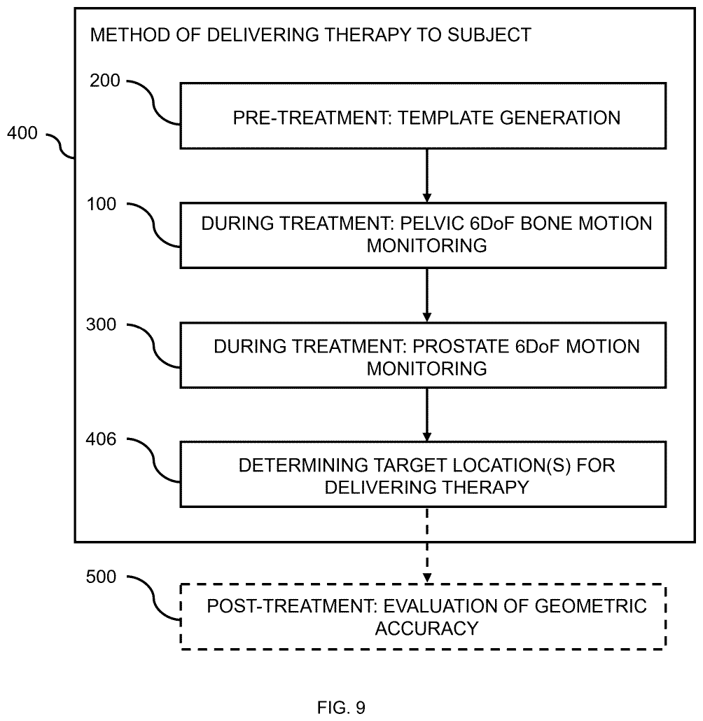

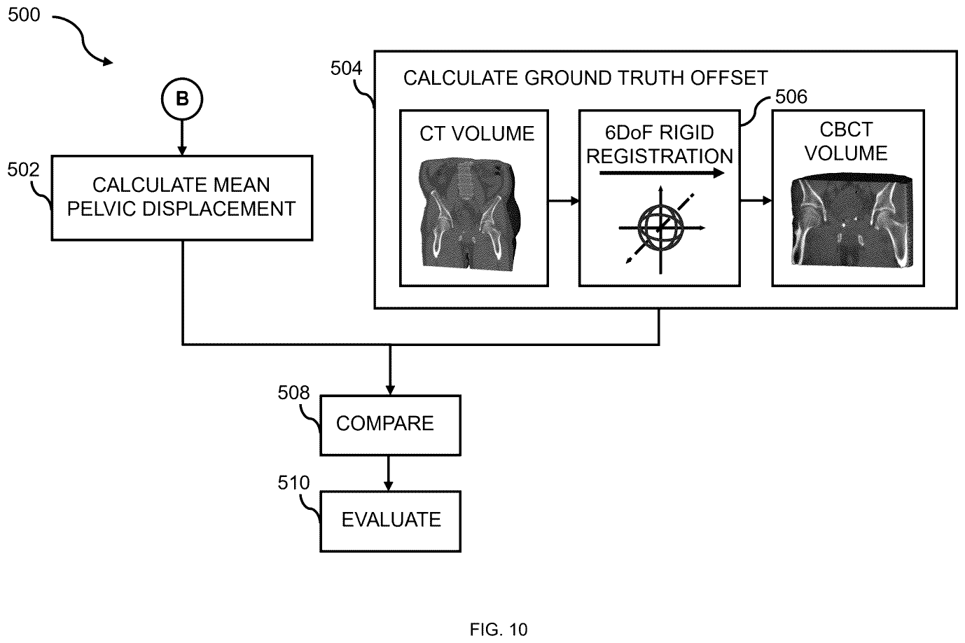

The method 100 as shown in FIG. 5 may finally include a step 130 of outputting displacement of the anatomical feature or pelvis 70 in six degrees of freedom (6DoF). The displacement is output for use in the method 500 of FIG. 10 of evaluating the geometric accuracy as indicated by the arrow with the encircled letter “B”. The displacement data may also be output for use in a method 400 for delivering therapy to a subject 50, where the motion data is provided for determining one or more locations for delivering therapy to the subject 50 (see FIGS. 8 and 9).

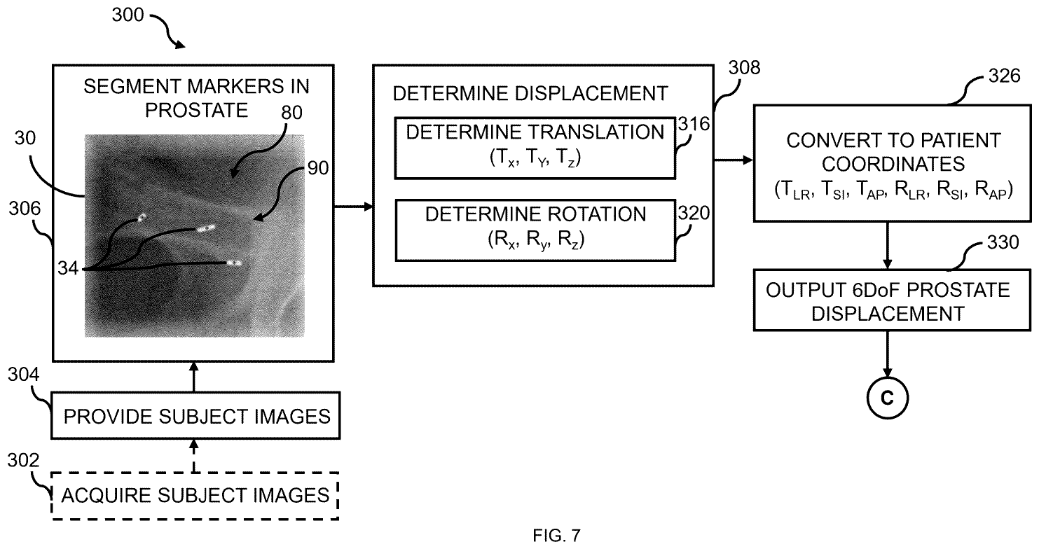

FIG. 7 is a schematic diagram illustrating a method 300 of monitoring motion of another anatomical feature 90 of the subject 50, in this case the prostate, and outputting the prostate displacement in six degrees of freedom (DoF), with optional steps shown in broken lines, according to some embodiments of the disclosure. Similar to the previous methods, the method 300 may optionally include the step 302 of acquiring the subject images 30 including the prostate 90, or otherwise providing the subject images 30 at step 304 ready for processing.

The method 300 outlines the steps in monitoring prostate motion according to the KIM method, in which fiducial markers 34 implanted in the prostate 90 are automatically segmented on the same kV image of the region 80 of the subject's body 60 as shown in step 306 and the displacement is determined at step 308, where the 3D motion in respect of translation and rotation are estimated based on a 3D Gaussian probability density function (PDF) at steps 316, 320 and 324. Rotation of the prostate 90 can be calculated using an iterative closest point algorithm for each fiducial marker 34 to calculate a rotation matrix^{20,27}.

The method 300 includes at step 308 determining displacement of the anatomical feature 90, e.g., the prostate, in six degrees of freedom (6DoF) comprising determining translation (Tₓ, T_{y}, T_{z}) at step 316 and determining rotation (Rₓ, R_{y}, R_{z}) at step 320 of the anatomical feature 90. The method 300 may further include the step 326 shown in FIG. 7 of converting the displacement to the patient coordinates. The step 326 may include applying a rotational transformation to determine the prostate displacement in the present example in the patient coordinates with respect to the planning CT. The displacement of the anatomical feature (Tₓ, T_{y}, T_{z}, Rₓ, R_{y}, R_{z}) may be transformed to a frame of reference of the subject's body (T_{LR}, T_{SI}, T_{AP}, R_{LR}, R_{SI}, R_{AP}).

The method 300 as shown in FIG. 7 may finally include a step 330 of outputting displacement of the anatomical feature or prostate 90 in six degrees of freedom (6DoF). The displacement may be output for use in the method 400 of FIG. 8 of delivering therapy to a subject 50 as indicated by the arrow with the encircled letter “C”. Motion data which includes the monitored displacement of the prostate 90 over a period of time may be provided for use in the method 400 at step 404. The motion data may include differences in the displacement of the prostate 90 over the period of time.

The steps of the method 300 may be performed by one or more processors of a processing unit 606 of a therapy delivery system 600 as shown in FIG. 11. Alternatively, the steps may be performed off-board and the processing unit 606 is configured to receive the motion data of the prostate 90 from a network 630 via a network adapter 628 as shown in FIG. 11.

FIG. 8 is another flow chart illustrating a method 400 of delivering therapy to a subject 50 according to some embodiments of the disclosure. The method 400 includes a first step 402 of providing motion data of a first anatomical feature 70 within a region 80 of the subject's body 60, where the motion data is determined according to the method 100 for monitoring motion of an anatomical feature 70 within the subject's body 60. The method 400 may optionally include the step 404 of providing motion data of a second anatomical feature 90 within the region 80 of the subject's body 60. The method 400 also includes the step 406 of determining one or more target locations within the region 80 of the subject's body 60 for delivering therapy based on the provided motion data of the first anatomical feature 70 and/or optionally the second anatomical feature 90. The method 400 also includes the step 408 of operating a therapy delivery system 600 (see FIG. 11) to deliver therapy to the subject 50 at the one or more target locations.

The motion data of the first anatomical feature 70 may be determined according to any one of the embodiments of the method 100 described in respect of FIGS. 3 to 7. The motion data of the first anatomical feature 70 may include differences in displacement of the first anatomical structure 70 over a time series of images 30 of the region 80 of the subject's body. The first anatomical feature 70 may be the pelvis of the subject 50. The motion data of the second anatomical feature 90 may be determined according to the method 300 for monitoring motion of an anatomical feature 90 within the subject's body. The motion data of the second anatomical feature 90 may include differences in displacement of the second anatomical feature 90 in images 30 of the region 80 of the subject's body 60 acquired over a period of time. The second anatomical feature 90 may be the prostate of the subject 50.

The step 406 of determining one or more target locations may comprise the step of determining one or more target locations for delivering therapy based on the motion data of the first anatomical feature 70 and the second anatomical feature 90. The step 406 of determining one or more target locations may comprise the step of determining a target location for delivering therapy to each of the first anatomical feature 70 and the second anatomical feature 90 based on the provided motion data of the first anatomical feature 70 and the second anatomical feature 90. The method may then include at step 408 of operating the therapy delivery system to simultaneously deliver therapy to the subject 50 at both target locations of the first anatomical feature 70 and the second anatomical feature 90.

In some embodiments, one or more steps of the method 400 are performed during delivery of therapy to the subject 50. The steps 402 and 404 of providing motion data may be performed during delivery of therapy to the subject 50 for monitoring in vivo motion of the first anatomical feature 70 and/or the second anatomical feature 90 within the subject's body 60, e.g., while the subject 50 is being treated. The step 406 of determining one or more target locations may also be performed during delivery of therapy to the subject 50. In some embodiments, one or more steps of the method 400 are also performed in real-time.

In a preferred embodiment, the first anatomical feature 70 is the pelvis of the subject 50, and the second anatomical feature 90 is the prostate of the subject 50. Thus, in embodiments of the disclosure, a method for delivering multi-targeted therapy to a patient may be provided, with optional simultaneous targeting of the pelvis and prostate of the patient, e.g., for cancer treatment.

The step 408 of operating the therapy delivery system 600 to simultaneously deliver therapy to the subject 50 at both target locations may be achieved with various implementations. Online adaptive radiotherapy strategies can be used to account for interfraction displacements that occur between primary tissue treatment targets and the associated anatomical features, e.g., lymph nodes, by generating a new treatment plan based on the anatomy seen in images acquired on the day of treatment^{12,39,40}. For example, multiple targets may be irradiated simultaneously by operating the therapy delivery system 600 with a modified aperture shape and segment weights for intensity-modulated radiation therapy plans according to relative interfraction shifts^{2,11,41,42}.

To adapt to intrafraction motion between multiple targets, real-time MLC tracking has been demonstrated to adapt the radiation beam to prostate and lymph node targets for patients with locally advanced prostate cancer^{15,43}. As multi-target MLC tracking can be implemented on standard linear accelerators¹⁶, it may be integrated with the multi-target prostate and pelvic bone KIM motion monitoring of embodiments of the present disclosure to allow for real-time multi-target adaptive radiation therapy delivery for locally advanced prostate cancer patients. Applying the real-time 6DoF bony anatomy targeting method 100 also be used for other sites, such as the spine or vertebrae, as the accurate delivery of radiation therapy to the spine is even more crucial. Spine position monitoring has been previously investigated during SBRT delivery, however has been limited to 3D motion^{44,45} or rotation only in the imaging plane⁴⁶.

FIG. 9 is another flow chart illustrating a method of delivering therapy to a subject 50, including the steps from the method of FIGS. 3 to 7 and determining target location(s) for delivering therapy, with optional post-treatment evaluation of geometric accuracy of the method shown in broken lines, according to some embodiments of the disclosure. The method 400 in this embodiment may include the methods 100, 200, 300 as previously described, namely the pre-treatment method 200 of template generation, the during treatment method 100 of monitoring motion of the pelvic bone in 6DoF, and the during treatment method 300 of monitoring motion of the prostate in 6DoF. The method 400 includes a further step 406 of determining one or more target location(s) for delivering therapy to the subject 50 based on the monitored motion data of the pelvic bone and prostate in 6DoF. The method 400 may also include any one of the steps described above with reference to FIG. 8.

FIG. 10 is a schematic diagram illustrating additional steps in the method of FIG. 9 relating to post-treatment evaluation of geometric accuracy, according to some embodiments of the disclosure. A method 500 of post-treatment evaluation of geometric accuracy includes, based on the pelvic bone 6DoF displacement output at step 130 of FIG. 5, the step of calculating the mean pelvic displacement. The method 500 may also include a step 504 of calculating a ground truth offset between the pelvic bone displacement during acquiring of pre-treatment cone-beam computed tomography (CBCT) images and in the planning CT. The ground truth offset is calculated at step 506 by performing an automatic 6DoF rigid registration of the planning CT to the 3D reconstructed CBCT. This may be achieved using existing image processing methods, including the Elastix toolbox²⁸.

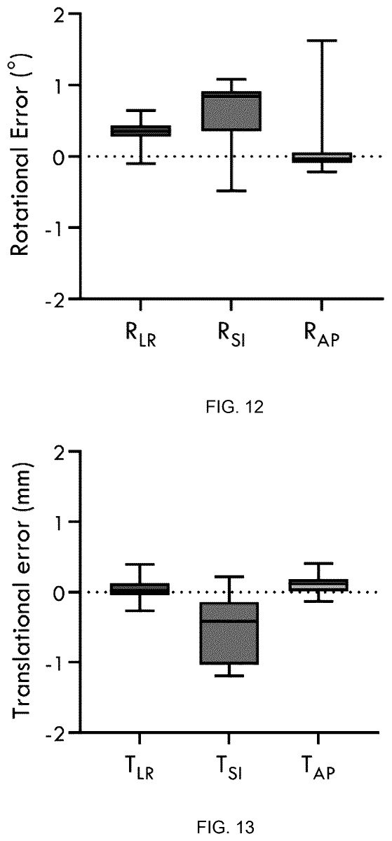

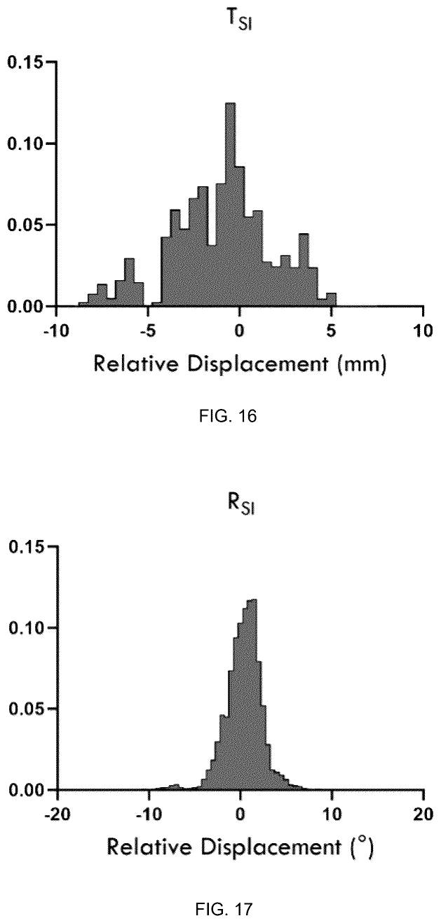

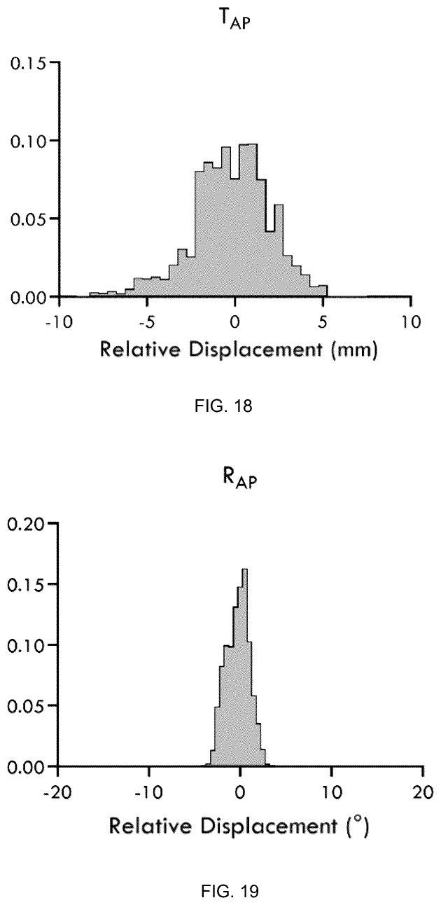

The method 500 includes a comparison step 508 between the mean pelvic displacement calculated at step 502 and the ground truth offset calculated at step 504. The geometric accuracy is then evaluated at step 510 based on the comparison at step 508. The results of the geometric accuracy of the pelvic motion monitoring method 100 will be described in relation to Example 1 and FIGS. 12 to 23.

The steps of the method 400 and 500 of FIGS. 8 to 10 may be performed by one or more processors of a processing unit 606 of a therapy delivery system 600 as shown in FIG. 11. Alternatively, the steps may be performed off-board and the processing unit 606 is configured to receive information for processing from a network 630 via a network adapter 628 as shown in FIG. 11.

Referring now to FIG. 11, a schematic of a therapy delivery system 600 according to another aspect of the present disclosure is shown. The therapy delivery system 600 comprises a therapy system 604 for delivery therapy to a subject 50. The system 600 also includes an imaging system 602 for imaging a region 80 of the subject's body 60. The system 600 also includes one or more processors 606 configured to provide motion data of a first anatomical feature 70 within the region 80 of the subject's body 60, the motion data being determined according to the method 100 as previously described, and determine one or more target locations within the region 80 of the subject's body 60 for delivering therapy based on the provided motion data of the first anatomical feature 70. The system 600 also includes a controller 608 configured to operate the therapy delivery system 600 to deliver therapy to the subject 50 at the one or more target locations.

In some embodiments, the one or more processors 606 may also be configured to provide motion data of a second anatomical feature 90 within the region 80 of the subject's body 60. The one or more processors 606 may be configured to determine one or more target locations within the region 80 of the subject's body 60 for delivering therapy based on the motion data of the first anatomical feature 70 and the second anatomical feature 90.

In some embodiments, the one or more processors 606 may be configured to determine a target location for delivering therapy to each of the first anatomical feature 70 and the second anatomical feature 90. The controller 608 may be configured to operate the therapy delivery 600, e.g., the therapy system 604, to deliver therapy to the subject 50 at the one or more target locations, and preferably, may delivery therapy simultaneously to each of the target locations for the first anatomical feature 70 and the second anatomical feature 90.

The one or more processors 606 may be configured to provide motion data of the first anatomical feature 70 and/or the second anatomical feature 90 during delivery of therapy to the subject 50 for monitoring in vivo motion of the first anatomical feature 70 and/or the second anatomical feature 90 within the subject's body 60. The one or more processors 606 may also be configured to determine one or more target locations during delivery of therapy to the subject 50. The one or more processors 606 may also be configured to provide motion data and determining one or more target locations in real time.

The therapy delivery system 600 may include similar hardware components to the therapy delivery system 10 as shown and described in relation to FIGS. 1 and 2. The therapy system 604 may include a rotatable linear accelerator supported by a gantry for delivering radiation therapy to the subject 50 (e.g., see linear accelerator 12 supported by gantry 26 in FIGS. 1 and 2). The imaging system 602 may include an on-board imaging system with a source and detector for x-ray imaging of the region 80 of the subject's body 60 (e.g., see imaging system 14 with source 16 and detector 18 in FIGS. 1 and 2). The therapy delivery system 600 may include a couch or tray for the patient to be positioned on in a supine position for treatment (e.g., see couch or tray 24 of FIGS. 1 and 2). The therapy delivery system 600 may also include a display 624 such as a display screen for displaying to an operator information regarding radiation therapy delivery and motion monitoring of the anatomical feature and treatment tissue. One or more external device(s) 626 may also be included in the system 600 to allow for the operator to control inputs and outputs of the therapy delivery system 600.

As shown in FIG. 11, the therapy delivery system 600 includes a computer system or server 620 with a processing unit or one or more processors 606 to determine target location(s) for delivering therapy and to output instructions to the controller 608 to operate the therapy system 604 to deliver therapy to the subject 50, according to some embodiments of the disclosure. In this embodiment, the computer system 620 and processing unit 606 are provided on-board the therapy delivery system 600. However, in other embodiments, the computer system 620 and/or the processing unit 606 may be provided off-board. The processing unit or processor 606 of may be used to implement certain steps of the methods 100, 200, 300, 400 and 500 of embodiments of the disclosure (see FIGS. 3 to 10) and performed in the functioning of the therapy delivery system 600. The processing unit or processor 606 may include a micro-processor configured to receive data or information from other components of the therapy delivery system 600 or a computing system or server 620, such as through a wireless or hard-wired connection (see network 630 and bus 632).

The controller 608 may be configured to operate the imaging system 602 and the therapy system 604. The controller 608 may include a programmable logic controller (PLC) and/or an embedded PCB (not shown). The controller 140 may contain or store a number of predefined instructions or steps in a non-volatile memory such as a hard drive. The controller 140 may be programmed by the operator of the therapy delivery system 600 to implement a number of steps of the methods 100, 200, 300, 400 and 500 of embodiments of the disclosure, or they may be predefined. The controller 608 and processor(s) 606 may include any other suitable controllers or processors known to a person skilled in the art. The steps performed by the processor(s) may be implemented through the controller 608 and further in software, firmware, and/or hardware in a variety of manners as would be appreciated by a person skilled in the art.

The computer system 620 may be operational with numerous other general purpose or special purpose computing system environments or configurations. Examples of well-known computing systems, environments, and/or configurations that may be suitable for use with computer system 620 include, but are not limited to, personal computer systems, server computer systems, handheld or laptop devices, multiprocessor systems, microprocessor-based systems, network PCs, minicomputer systems, mainframe computer systems, and distributed cloud computing environments that include any of the above systems or devices, and the like.

Computer system 620 may be described in the general context of computer system executable instructions, such as program modules, being executed by a computer system. Generally, program modules may include routines, programs, objects, components, logic, data structures, and so on that perform particular tasks or implement particular abstract data types. Computer system/server 620 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 11, computer system 620 in the therapy delivery system 600 is shown in the form of a general-purpose computing device. The components of computer system/server 620 may include, but are not limited to, one or more processors or processing units 606, a system memory 612, and a bus 632 that couples various system components including system memory 612 to processor 606.

Bus 632 represents one or more of any of several types of bus structures, including a memory bus or memory controller, a peripheral bus, an accelerated graphics port, and a processor or local bus using any of a variety of architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 620 typically includes a variety of computer system readable media. Such media may be any available media that is accessible by computer system/server 620, and it includes both volatile and non-volatile media, as well as removable and non-removable media.

System memory 612 can include computer system readable media in the form of volatile memory, such as random access memory (RAM) 614 and/or cache memory 616. Computer system/server 620 may further include other removable/non-removable, volatile/non-volatile computer system storage media. By way of example only, storage system 618 can be provided for reading from and writing to a non-removable, non-volatile magnetic media (not shown and typically called a “hard drive”), and other non-removable, non-volatile media (e.g., a “solid-state drive”). Although not shown, a magnetic disk drive for reading from and writing to a removable, non-volatile magnetic disk (e.g., a “floppy disk”), and an optical disk drive for reading from and/or writing to a removable, non-volatile optical disk such as a CD-ROM, DVD-ROM or other optical media can be provided. In such instances, each can be connected to a bus 632 by one or more data media interfaces. As will be further described below, memory 612 may include a computer program product storing a set (e.g., at least one) of program modules 611 comprising computer readable instructions configured to carry out one or more features of the present disclosure.

Program 610, having a set (at least one) of program modules 611, may be stored in memory 612 by way of example, and not limitation, as well as an operating system, one or more application programs, other program modules, and program data. Each of the operating system, one or more application programs, other program modules, and program data or some combination thereof, may include an implementation of a networking environment. In some embodiments, program modules 611 are adapted to generally carry out the one or more functions and/or methodologies of one or more embodiments.

Computer system 620 may also communicate with one or more external devices 626 such as a keyboard, a pointing device, a display 624, etc.; one or more devices that enable an operator to interact with computer system 620 or therapy delivery system 600; and/or any device (e.g., network card, modem, etc.) that enable computer system 620 to communicate with one or more other computing devices. Such communication can occur via Input/Output (I/O) interfaces 622. The computer system 620 can communicate with one or more networks such as a local area network (LAN), a general wide area network (WAN), and/or a public network (e.g., the Internet) via network adapter 628. As depicted, the network adapter 628 communicates with other components of computer system 620 via bus 632. It should be understood that although not shown, other hardware and/or software components could be used in conjunction with computer system 620. Examples include, but are not limited to: microcode, device drivers, redundant processing units, external disk drive arrays, RAID systems, tape drives, and data archival storage systems, etc.

In another aspect, the present disclosure provides a computer program product for monitoring motion of an anatomical feature 70 within a subject's body 60. The computer program product comprises a non-transitory computer readable storage medium 612 having program code 610 embodied therewith. The program code 610 is executable by one or more processors 606 to perform the method 100 for monitoring motion of an anatomical feature 70 within a subject's body 60. The method 100 comprises the step 104 of providing a time series of subject images 30 of a region 80 of the subject's body 60 comprising the anatomical feature 70. The method 100 also comprises the step 106 of providing a plurality of reference images 40 of the region 80 of the subject's body 60. The plurality of reference images 40 comprise a range of projection angles around an imaging axis (z-axis) and perpendicular to the imaging axis (x-axis) through the region 80 of the subject's body 60. The method 100 also comprises the step 108 of determining a displacement of the anatomical feature 70 in each subject image 30 using the plurality of reference images 40. The method 100 also comprises the step 110 of monitoring motion of the anatomical feature 70 based on the displacement of the anatomical feature 70 over the time series of subject images 30.

In some embodiments, the method 100 performed by the one or more processors 606 may include one or more of the steps previously described in respect of the methods 100, 200, 300, 400 and 500 of the embodiments of FIGS. 3 to 10.

In another aspect, the present disclosure provides a computer program product for delivering therapy to a subject 50. The computer program product comprises a non-transitory computer readable storage medium 612 having program code 610 embodied therewith. The program code 610 is executable by one or more processors 606 to perform the method 400 of delivering therapy to a subject 50. The method 400 comprises the step 402 of providing motion data of a first anatomical feature 70 within a region 80 of the subject's body 60, the motion data being determined according to the method 100 of monitoring motion of an anatomical feature 70 (see FIGS. 3 to 6). The method 400 also optionally comprises the step 404 of providing motion data of a second anatomical feature 90 within the region 80 of the subject's body 60 (e.g., by the method 300 of FIG. 7). The method 400 also comprises the step 406 of determining one or more target locations within the region 80 of the subject's body 60 for delivering therapy based on the provided motion data of the first anatomical feature 70 and optionally, the second anatomical feature 90. The method 400 also comprises the step 408 of operating a therapy delivery system 600 to deliver therapy to the subject 50 at the one or more target locations.

In some embodiments, the method 400 performed by the one or more processors 606 may include one or more of the steps previously described in respect of the methods 100, 200, 300, 400 and 500 of the embodiments of FIGS. 3 to 10.

The computer readable storage medium 612 can be a tangible device that can retain and store instructions for use by an instruction execution device, such as a memory device shown in FIG. 11. The computer readable storage medium 612 may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium 612 includes the following: a portable computer diskette, a hard disk, a random access memory (RAM) 614, a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices 606 from a computer readable storage medium 612 or to an external computer or external storage device via a network 630, for example, the Internet, a local area network, a wide area network and/or a wireless network. The network 630 may comprise copper transmission cables, optical transmission fibers, wireless transmission, routers, firewalls, switches, gateway computers and/or edge servers. A network adapter card or network interface 628 in each computing/processing device 606 receives computer readable program instructions from the network 630 and forwards the computer readable program instructions for storage in a computer readable storage medium 612 within the respective computing/processing device 606.

Computer readable program instructions for carrying out operations may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the operator's computer, partly on the operator's computer, as a stand-alone software package, partly on the operator's computer and partly on a remote computer or entirely on the remote computer or server. In the latter scenario, the remote computer may be connected to the operator's computer through any type of network, including a local area network (LAN) or a wide area network (WAN), or the connection may be made to an external computer (for example, through the Internet using an Internet Service Provider). In one or more embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects are described herein with reference to flowchart illustrations and/or block diagrams of methods, systems, and computer program products according to one or more embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that various modifications, additions and/or alternatives may be made to the parts previously described without departing from the ambit of the present invention as defined in the claims appended hereto.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

### EXAMPLE

An example illustrating an application of some embodiments of the disclosure will now be described. Example 1 is supplied to provide context and explain features and advantages of embodiments of the disclosure and is not limiting on the scope of the invention as defined in the claims.

Method. A method to track the pelvic bone translation (T_{LR}, T_{SI}, T_{AP}) and rotation (R_{LR}, R_{SI}, R_{AP}) in intrafraction kV images was performed according to the method 100 of monitoring motion of an anatomical feature 70 within a subject's body 60 of embodiments of the disclosure as previously described. The method was tested retrospectively on images collected from 20 prostate cancer patients who were treated in the Trans-Tasman Radiation Oncology Group (TROG) 15.01 Stereotactic Prostate Ablative Radiotherapy with KIM (SPARK) trial²¹. The anonymized patient images are publicly available²⁴.

1. Pre-treatment template generation. A plurality of reference images 40 or patient library were generated according to the method 200 previously described in relation to FIG. 4. To generate a template of the anatomy of interest, the pelvic bone on each patient's planning CT was contoured and used to mask the CT volume. The femurs were excluded from the contour. Digitally reconstructed radiographs (DRRs) were generated of the masked CT volumes using the open-source Reconstruction Toolkit (RTK) 25. As the expected shape of the pelvic bone on the 2D treatment images would vary depending on the gantry angle, DRRs at a range of projection angles were generated to form a 6DoF library for each patient. DRRs were generated for angles around the superior-inferior (R_{SI}) axis ranging from 0° to 359.5° with intervals of 0.5°. For each R_{SI} angle, projections with the pelvic bone rotating out of the imaging plane (Rₓ) were also generated, ranging from −6° to +6° with intervals of 0.5°. The magnitude of the gradients for each DRR in the x and y directions were then computed so that the edges of the bony anatomy would form the dominant feature in each template.

2. 6DoF motion monitoring of the pelvic bone. The pelvic bone was monitored according to the method 100 previously described in relation to FIGS. 5 and 6. Pelvic bone segmentation was performed on kV images 30 acquired with a known gantry angle. The method implemented three-frame temporal averaging and a median filter to reduce image noise, followed by a Gaussian bandpass filter to enhance the edges of the bony anatomy in the image 30. The fiducial markers 34 implanted in the prostate 90 for prostate motion monitoring were automatically masked in the kV images 30. The gradients of each kV image 30 were calculated to generate an image that highlighted the edges of the bony anatomy, consistent with the method 200 used for template generation.

Template matching was performed by calculating the 2D normalized cross-correlation of the template and the kV treatment image according to the method as previously described in relation to FIG. 6. The x and y values that corresponded to the maximum normalized cross-correlation coefficient were used to determine the 2D displacement (Tₓ and T_{y}) of the pelvic bone 70 during treatment from the planning CT. The search region for Tₓ and T_{y} was limited to 1 mm in each direction from the preceding images. This calculation was repeated for a selection of DRRs from the generated library, to check rotations close to the expected rotation of the pelvis in the Rₓ and R_{y} directions. Each template image 40 was also rotated around the imaging axis (z-axis) to calculate the normalized cross-correlation for varying R_{z} rotations. The DRR and R_{z} rotation that had the highest normalized cross-correlation value were used to determine the magnitude of Rₓ, R_{y}, and R_{z}. For the first treatment image, rotations ranging ±6° from the planned pelvic bone position were tested, and for subsequent images rotations ranging ±0.5° from the previous image were tested.

The final unresolved motion, translation along the imaging axis (T_{z}), was calculated using a probability-based method previously described by Poulsen et al²⁶. This method assumes a 3D Gaussian probability density function (PDF) for target position. An initial PDF was built using the position of the pelvis observed in the first 20 projections acquired using maximum likelihood estimation. Then, the unknown position of the pelvis along the direction of the imager axis was estimated by finding the expectation value determined by the 1D Gaussian distribution along the imaging axis.

Finally, a rotational transformation was applied to determine the pelvic bone displacement in the patient coordinates (T_{LR}, T_{SI}, T_{AP} and RER, R_{SI}, R_{AP}) with respect to the planning CT.

3. Prostate motion was monitored using KIM, which automatically segmented fiducial markers 34 implanted in the prostate 90 on the same kV image 32 and estimated the 3D motion based on a 3D Gaussian PDF. Rotation of the prostate 90 was then calculated using an iterative closest point algorithm for each marker to calculate a rotation matrix^{20,27}. The steps in the method 300 of motion monitoring of the prostate 90 were previously described in relation to FIG. 7.

4. Retrospective geometric accuracy analysis. The analysis was performed according to the method 500 previously described in relation to FIG. 10. To evaluate the geometric accuracy of the pelvic tracking method described above, the method was tested retrospectively on prostate patient data. The patients included in Example 1 were treated using a Varian TrueBeam linear accelerator, at three different treatment institutions. Two-dimensional images acquired using the on-board kV imaging system on the linear accelerator were tested for 20 patients, who were each treated in five fractions. Each patient was treated using stereotactic body radiation therapy (SBRT) guided by KIM to provide real-time 6DoF motion of the prostate²³.

Due to the limited field of view of the intrafraction KIM images, the multi-target motion monitoring method was tested on 2D kV projections acquired for the pre-treatment cone-beam computed tomography (CBCT) image at the beginning of each treatment to guide the initial patient set-up. These images were acquired over a 200° arc at a rate of 14 Hz, with an average of 470 full-fan projections acquired per CBCT. The projections had an average angle separation of 0.4°. The imaging system had a source to-axis distance (SAD) of 1000 mm and a source-to-detector distance (SDD) of 1500 mm. Each image was acquired with a resolution of 1024×774 pixels and a pixel size of 0.388×0.388 mm², and then cropped to a field size of 180×180 mm² for pelvic motion monitoring.

The ground truth offset between the pelvic bone displacement during CBCT acquisition and in the planning CT was evaluated by performing an automatic 6DoF rigid registration of the planning CT to the 3D reconstructed CBCT using the Elastix toolbox²⁸. The planning CTs were acquired with 1.5 mm, 2 mm, and 2.5 mm slice thicknesses depending on the treatment institution. Both the translational and rotational displacements were calculated and compared to the output of the pelvic motion tracking method. The rationale for choosing this ground truth is that the mean of the real-time 6DoF tracking during the image acquisition should equal the reconstructed image registration, assuming that the means from these two processes should be similar.

Results. The results will now be shown and described in relation to FIGS. 12 to 23.

1. 6DoF pelvic motion monitoring accuracy. The overall geometric accuracy of the pelvic motion monitoring method with reference to the patients' coordinate system is shown in FIGS. 12 and 13. The geometric error of the pelvic tracking motion monitoring method for translation and rotation in the patient coordinate system for 20 patients across five fractions. The whiskers represent the minimum and maximum values. The mean and standard deviation geometric errors for the translational directions T_{LR}, T_{SI}, and T_{AP} were 0.0±0.1 mm, −0.5±0.5 mm, and 0.1±0.1 mm respectively, and for the rotational directions R_{LR}, R_{SI}, and R_{AP} were 0.3° ±0.1°, 0.6°±0.4°, and 0.0°±0.3° respectively.

The geometric accuracy of prostate motion monitoring using KIM for this cohort of patients has been previously reported to be sub-mm for translation and within 1.4° for rotation²³.

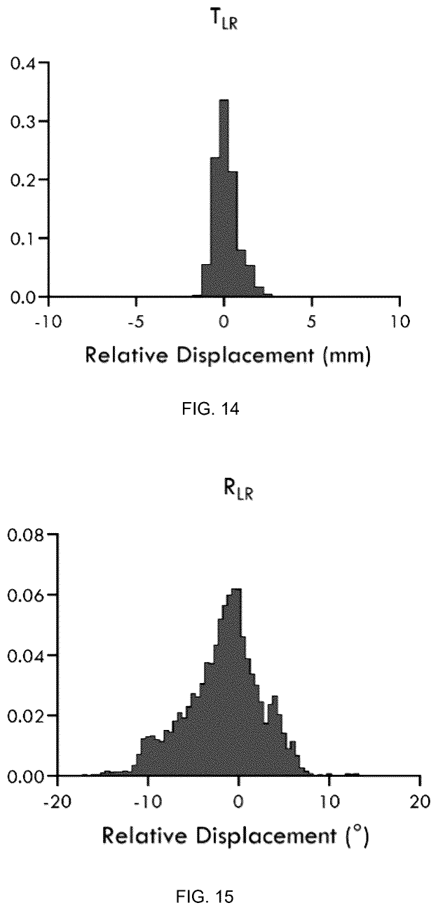

2. 6DoF multi-target displacements. The distribution of relative displacements between the pelvic bone and prostate observed in the kV projections is shown in FIGS. 14 to 19. Histograms of the relative translational (top row) and rotational (bottom row) displacements observed between the pelvic bone and prostate across all kV images for the 20 patients analyzed in Example 1.

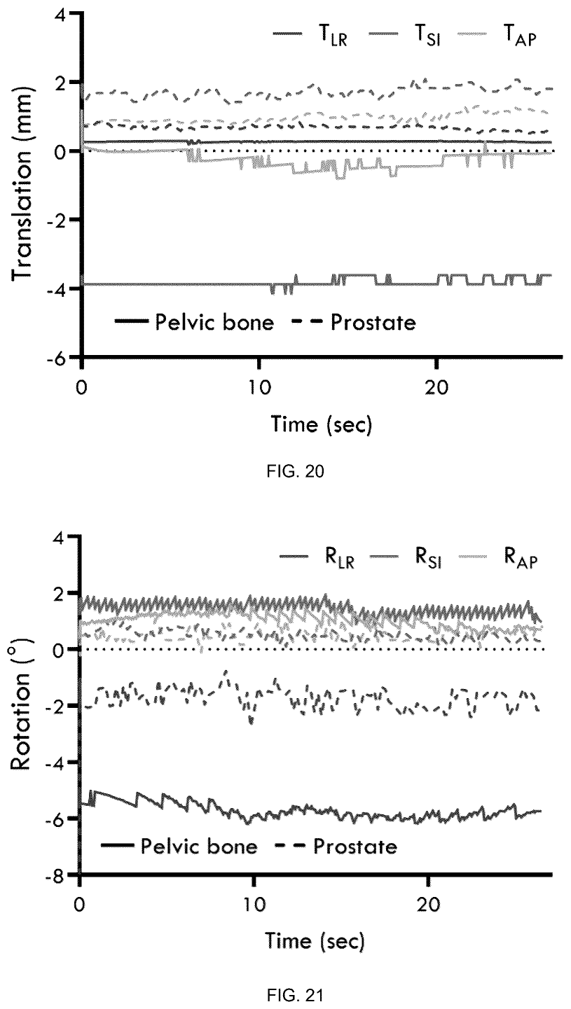

The 5^{th} and 95^{th} percentiles for the relative motion between the prostate and pelvic bone were [−0.8 mm, 1.4 mm], [−6.2 mm, 3.6 mm], and [−4.2 mm, 3.2 mm] for the T_{LR}, T_{SI}, and T_{AP} directions, and [−9.7°, 4.8°], [−2.9°, 3.2°], and [−2.4°, 1.8°] for the R_{LR}, R_{SI}, and R_{AP} directions respectively. The 3D relative displacement between the prostate and pelvic bone exceeded 2 mm, 3 mm, 5 mm, and 7 mm for approximately 66%, 44%, 12%, and 7% of the time respectively. Correlation of motion between the pelvic bone and prostate was small for both translation (ρ<0.3) and rotation (ρ<0.4) in all three directions. An example of the relative motion observed for one patient is shown in FIGS. 19 and 20. FIGS. 19 and 20. The translation and rotation observed for the pelvic bone (solid lines) and prostate (dashed lines) for one patient. The ground truth displacement for the pelvic bone calculated from the rigid 6DoF registration of the CT to the CBCT was 0.3 mm, −2.9 mm, and −0.5 mm in the T_{LR}, T_{SI}, and T_{AP} directions, and −6.1°, 0.5°, and 0.5° in the R_{LR}, R_{SI}, and R_{AP} directions.

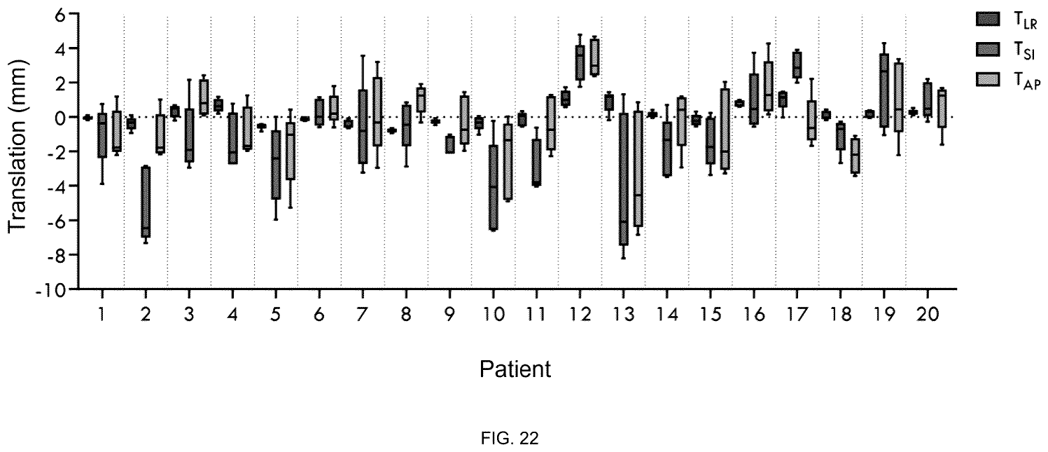

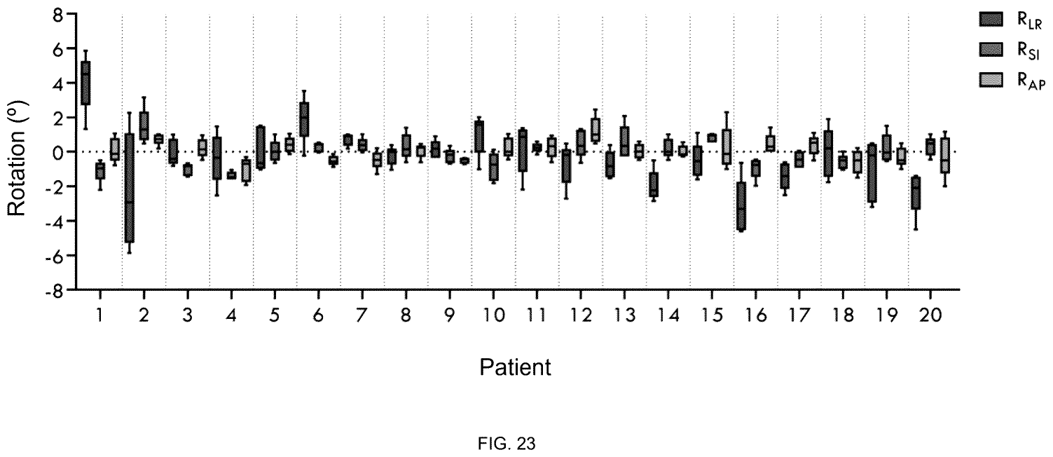

3. Resulting interfraction displacements. After each CBCT scan was acquired during the patient's treatment, a translational couch shift was applied to set the patient up to correctly align the prostate to isocenter for the beginning of treatment. Rotation was not corrected during patient setup. The resulting interfraction displacements of the pelvic bone for translation and rotation after patient setup for each patient across five fractions are shown in FIGS. 22 and 23.

Discussion. The present disclosure and Example 1 present an intrafraction pelvic motion monitoring method 100 and therapy delivery method 400 that allows for simultaneous motion monitoring of the skeletal anatomy as a surrogate for the pelvic lymph nodes and the implanted prostate markers, using kV images acquired on a standard linear accelerator during treatment. A template matching method 200 that utilized a pre-generated library of DRRs containing projections of the pelvic bone simulating a range of projection angles to allow for estimation of the pelvic bone translation and rotation was developed and evaluated. The pelvic motion monitoring method 100 was found to have a geometric accuracy and precision within 1 mm and 1° for images acquired for prostate cancer patients treated as part of the TROG 15.01 SPARK trial.

Relative motion between the pelvic bone anatomy and the prostate was also reported. Relative translations between the pelvic bone and prostate were largest in the T_{SI} and T_{AP} directions, while rotations were largest around the R_{LR} axis (pitch). This finding is consistent with previous observations of internal prostate motion^{7,29,30}. The observed relative motions between the pelvic bone and prostate suggest that when motion management is based on the prostate, a 5 mm margin would be sufficient for the pelvic lymph nodes 88% of the time. However, it should be noted Example 1 was limited to measuring the relative motion between the prostate and pelvic lymph nodes within a short window throughout the acquisition of the pre-treatment CBCT, and larger displacements could be observed when the entire fraction is considered. Tyagi et al.³¹ similarly examined the relative differences between patient setup based on a fiducial match and bony anatomy match for 30 patients receiving SBRT to the prostate and pelvic lymph nodes. Larger translational shifts were observed in the study by Tyagi et al., where a 5 mm shift would have only covered approximately 75% of patients, and 19% of fractions would have seen a significant loss in dosimetric coverage to the pelvic lymph nodes. Pelvic bone rotations were also measured in Example 1, with all pelvic rotations in the R_{SI} and R_{AP} directions being within 3°, and rotations in the R_{LR} direction were within 6°. Larger rotations were seen for the prostate compared to the pelvis and were independent of the pelvic rotations.

An alternative method of estimating 6DoF pelvic bone pose on 2D images has been demonstrated by Munbodh et al^{32,33}. Instead of relying on a library of DRRs with a predetermined set of pelvic rotations, 2D DRRs were computed after iteratively performing rigid spatial transformations of the CT, optimizing for the translation and rotation parameters using a gradient ascent search strategy. While this approach prioritized establishing a high registration accuracy, the computation time to achieve a single registration solution would not be able to be applied to intrafraction monitoring of the pelvic bone position. Similar methods relying on fast generation of DRRs have also been applied to verify the 6DoF position of other structures such as the spine³⁴ or cranium³⁵. Registration of 2D to 3D images to verify patient setup has also been performed by acquiring orthogonal 2D projections, however the acquisition of images at separate gantry angles on a standard linear accelerator will involve a time delay³⁶.

The method demonstrated by Munbodh et al^{32,33} and others does not contemplate or even consider acquiring images at separate gantry angles prior to treatment to provide a library of DRRs to be matched to the images acquired during treatment. Furthermore, none of the existing methods provide for estimation of translation motion in the unresolved direction along the z-axis (T_{z}) according to embodiments of the present disclosure. Munbodh et al^{32,33} use image magnification to estimate the translational offset along this direction while the method of the present disclosure employs a probability-based method described in more detail by Poulson et al²⁶.

The range of pelvic bone rotations that can be estimated is limited by the DRR library that is generated in the method 200 before treatment in the present disclosure. In Example 1, projections with a range of rotations of the pelvis in the Rₓ and R_{y} directions were generated with 0.5° intervals, limiting the precision with which the rotation in these directions could be estimated. In addition, only pelvic rotations in the Rₓ direction ranging from +6° were considered in the DRR library, so pelvic rotations larger than this around the Rₓ axis could not be measured in the presented implementation. The search area for consecutive images was also limited to 1 mm and 0.5° which would limit detection of large, abrupt motion. While DRR libraries with a higher angle resolution and wider range of rotations can be generated to increase the domain of pelvic poses that can be precisely estimated, this would come at the cost of longer DRR generation times and larger memory requirements to load the DRR library at the time of treatment for fast template access.

Integrating pelvic bone motion monitoring method 100 with the current KIM method 300 allows for simultaneous motion monitoring for both the prostate and lymph node targets during radiation therapy treatment as per the method 400. KIM was found to have a geometric accuracy and precision of 0.0 mm+0.4 mm, 0.1 mm+0.3 mm, 0.0 mm+0.5 mm in the T_{LR}, T_{SI}, and T_{AP} directions, and −0.1°+1.4°, −0.1°+1.0°, −0.1°+0.6° in the R_{LR}, R_{SI}, and R_{AP} directions respectively for prostate motion monitoring in the TROG 15.01 SPARK trial²³. Thus, a combination of the methods would be able to achieve both prostate and pelvic bone motion monitoring to within 0.5 mm and 1.4°. However, it should be noted that while patient alignment strategies assume that the pelvic lymph nodes are fixed to the bony anatomy and therefore the pelvic bone is a suitable surrogate for pelvic bone motion^{8,12,37}, magnetic resonance imaging (MRI) studies have indicated that there can be mobility of the lymph nodes relative to the bones with mean absolute deviations of up to 1.1 mm, 3.3 mm, and 2.1 mm in the LR, AP, and SI directions³⁸. Given the low soft tissue contrast in x-ray images compared to MRI, the pelvic bone position currently provides the best estimate for the pelvic lymph nodes on standard x-ray guided linacs, but lymph nodes could potentially be tracked more accurately on MR-linac systems.

Embodiments of the present disclosure provide a method to allow for displacement of the pelvic bone to be monitored simultaneously with prostate motion in 6DoF using 2D kV images. In Example 1, the method was retrospectively applied to data acquired during patient treatment from the TROG 15.01 SPARK trial and sub-mm and sub-degree geometric accuracy and precision of pelvic bone tracking was demonstrated. Advantageously, the integration of an intrafraction pelvic bone motion monitoring method according to embodiments of the present disclosure with prostate tracking could enable image guided real-time multi-target adaptation to occur during radiation therapy for patients with locally advanced disease.

Multi-target motion monitoring of embodiments of the present disclosure could be expanded into monitoring multiple targets for other anatomical sites. The seminal vesicles are typically included in the target volume for prostate cancer patients, however deformations resulting in relative motion between the prostate and the seminal vesicles are known to occur⁴⁷⁻⁴⁹. This motion is not monitored during treatments with the combined volume instead being treated as being rigid, requiring relatively large PTV margins to be used. Relative displacements of targets are also a known problem for lung^{50,51} and oligometastatic patients⁵². The increase in availability of combined MR-linac systems^{53,54} could lead to an improvement in capabilities to simultaneously monitor multiple targets during radiation therapy as well as the motion of nearby organs-at-risk to guide further dose-avoidance during treatment.

It is to be understood that the following claims are provided by way of example only, and are not intended to limit the scope of what may be claimed in any such future application. Features may be added to or omitted from the claims at a later date so as to further define or re-define the invention or inventions.

### REFERENCES

ADDIN E N.REFLIST 1. van Elmpt W, Öllers M, Lambin P, De Ruysscher D. Should Patient Setup in Lung Cancer Be Based on the Primary Tumor? An Analysis of Tumor Coverage and Normal Tissue Dose Using Repeated Positron Emission Tomography/Computed Tomography Imaging. International Journal of Radiation Oncology*Biology*Physics. 2012 Jan. 1/2012; 82 (1): 379-385. doi: https://doi.org/10.1016/j.ijrobp.2010.09.016
2. Hwang A B, Chen J, Nguyen T B, Gottschalk A G, Roach Iii M R, Pouliot J. Irradiation of the prostate and pelvic lymph nodes with an adaptive algorithm. https://doi.org/10.1118/1.3679859. Medical Physics. 2012 Feb. 1 2012; 39 (2): 1119-1124. doi: https://doi.org/10.1118/1.3679859
3. Kershaw L, van Zadelhoff L, Heemsbergen W, Pos F, van Herk M. Image Guided Radiation Therapy Strategies for Pelvic Lymph Node Irradiation in High-Risk Prostate Cancer: Motion and Margins. International Journal of Radiation Oncology*Biology*Physics. 2018 Jan. 1/2018; 100 (1): 68-77. doi: https://doi.org/10.1016/j.ijrobp.2017.08.044
4. Wang Z, Wang K, Lerma F A, et al. Planning Margins to CTV for Image-Guided Whole Pelvis Prostate Cancer Intensity-Modulated Radiotherapy. International Journal of Medical Physics, Clinical Engineering and Radiation Oncology. 2012; Vol.01No.02:9. 21717. doi: 10.4236/ijmpcero.2012.12004
5. Chung H T, Xia P, Chan L W, Park-Somers E, Roach M. Does Image-Guided Radiotherapy Improve Toxicity Profile in Whole Pelvic-Treated High-Risk Prostate Cancer? Comparison Between I G-IMRT and IMRT. International Journal of Radiation Oncology*Biology*Physics. 2009 Jan. 1/2009; 73 (1): 53-60. doi: https://doi.org/10.1016/j.ijrobp.2008.03.015
6. Roach M, DeSilvio M, Valicenti R, et al. Whole-pelvis, “mini-pelvis,” or prostate-only external beam radiotherapy after neoadjuvant and concurrent hormonal therapy in patients treated in the Radiation Therapy Oncology Group 9413 trial. International Journal of Radiation Oncology*Biology*Physics. 2006 Nov. 1/2006; 66 (3): 647-653. doi: https://doi.org/10.1016/j.ijrobp.2006.05.074
7. Langen K M, Willoughby T R, Meeks S L, et al. Observations on real-time prostate gland motion using electromagnetic tracking. International Journal of Radiation Oncology* Biology* Physics. 2008;71 (4): 1084-1090.
8. Shih H A, Harisinghani M, Zietman A L, Wolfgang J A, Saksena M, Weissleder R. Mapping of nodal disease in locally advanced prostate cancer: Rethinking the clinical target volume for pelvic nodal irradiation based on vascular rather than bony anatomy. International Journal of Radiation Oncology*Biology*Physics. 2005 Nov. 15/2005;63 (4): 1262-1269. doi: https://doi.org/10.1016/j.ijrobp.2005.07.952
9. Hsu A, Pawlicki T, Luxton G, Hara W, King C R. A Study of Image-Guided Intensity-Modulated Radiotherapy With Fiducials for Localized Prostate Cancer Including Pelvic Lymph Nodes. International Journal of Radiation Oncology*Biology*Physics. 2007 Jul. 1/2007; 68 (3): 898-902. doi: https://doi.org/10.1016/j.ijrobp.2007.02.030
10. Hinton B K, Fiveash J B, Wu X, Dobelbower M C, Kim R Y, Jacob R. Optimal planning target volume margins for elective pelvic lymphatic radiotherapy in high-risk prostate cancer patients. International Scholarly Research Notices. 2013; 2013
11. Ludlum E, Mu G, Weinberg V, Roach Iii M, Verhey L J, Xia P. An algorithm for shifting MLC shapes to adjust for daily prostate movement during concurrent treatment with pelvic lymph nodesa). https://doi.org/10.1118/1.2804579. Medical Physics. 2007 Dec. 1 2007; 34 (12): 4750-4756. doi: https://doi.org/10.1118/1.2804579
12. Ferjani S, Huang G, Shang Q, et al. Alignment Focus of Daily Image Guidance for Concurrent Treatment of Prostate and Pelvic Lymph Nodes. International Journal of Radiation Oncology*Biology*Physics. 2013 Oct. 1/2013; 87 (2): 383-389. doi: https://doi.org/10.1016/j.ijrobp.2013.06.003
13. Christiansen R L, Dysager L, Hansen C R, et al. Online adaptive radiotherapy potentially reduces toxicity for high-risk prostate cancer treatment. Radiotherapy and Oncology. 2022 Feb. 1/2022; 167:165-171. doi: https://doi.org/10.1016/j.radonc.2021.12.013
14. Tong X, Chen X, Li J, et al. Intrafractional prostate motion during external beam radiotherapy monitored by a real-time target localization system. Journal of Applied Clinical Medical Physics. 2015;16 (2): 51-61. doi: https://doi.org/10.1120/jacmp.v1612.5013
15. Hewson E A, Ge Y, O'Brien R, et al. Adapting to the motion of multiple independent targets using multileaf collimator tracking for locally advanced prostate cancer: Proof of principle simulation study. Medical Physics. 2021; 48 (1): 114-124. doi: https://doi.org/10.1002/mp.14572
16. Hewson E A, Dipuglia A, Kipritidis J, et al. First experimental evaluation of multi-target multileaf collimator tracking during volumetric modulated arc therapy for locally advanced prostate cancer. Radiotherapy and Oncology. 2021; 160:212-220.
17. Kilby W, Dooley J, Kuduvalli G, Sayeh S, Maurer Jr C. The CyberKnife® robotic radiosurgery system in 2010. Technology in cancer research & treatment. 2010; 9 (5): 433-452.
18. Ferris W S, Kissick M W, Bayouth J E, Culberson W S, Smilowitz J B. Evaluation of radixact motion synchrony for 3D respiratory motion: Modeling accuracy and dosimetric fidelity. Journal of Applied Clinical Medical Physics. 2020; 21 (9): 96-106.
19. Keall P J, Aun Ng J, O'Brien R, et al. The first clinical treatment with kilovoltage intrafraction monitoring (KIM): A real-time image guidance method. https://doi.org/10.1118/1.4904023. Medical Physics. 2015 Jan. 1 2015;42 (1): 354-358. doi: https://doi.org/10.1118/1.4904023
20. Huang C-Y, Tehrani J N, Ng J A, Booth J, Keall P. SixDegrees-of-Freedom Prostate and Lung Tumor Motion Measurements Using Kilovoltage Intrafraction Monitoring. International Journal of Radiation Oncology*Biology*Physics. 2015 Feb. 1/2015;91 (2): 368-375. doi: https://doi.org/10.1016/j.ijrobp.2014.09.040
21. Keall P, Nguyen D T, O'Brien R, et al. Stereotactic prostate adaptive radiotherapy utilising kilovoltage intrafraction monitoring: the TROG 15.01 SPARK trial. BMC Cancer. 2017 Mar. 8 2017;17 (1): 180. doi: 10.1186/s12885-017-3164-1
22. Lee YYD, Nguyen D T, Moodie T, et al. Study protocol of the LARK (TROG 17.03) clinical trial: a phase I I trial investigating the dosimetric impact of Liver Ablative Radiotherapy using Kilovoltage intrafraction monitoring. BMC Cancer. 2021 May 3 2021; 21 (1): 494. doi: 10.1186/s12885-021-08184-x
23. Hewson E A, Nguyen D T, O'Brien R, et al. The accuracy and precision of the KIM motion monitoring system used in the multi-institutional TROG 15.01 Stereotactic Prostate Ablative Radiotherapy with KIM (SPARK) trial. Medical Physics. 2019; 46 (11): 4725-4737. doi: https://doi.org/10.1002/mp.13784
24. Keall P N, Doan; O'Brien, Ricky; Hewson, Emily; Ball, Helen; Booth, Jeremy; Greer, Peter; Moodie, Trevor; Hardcastle, Nick; Poulsen, Per; Arumugam, Sankar; Martin, Jarad; Kneebone, Andrew; Siva, Shankar; Tai, Keen-Hun; Turner, Sandra; Eade, Thomas; Hayden, Amy; Sidhom, Mark; Hruby, George; Hunter, Perry; Wilton, Lee; Bromley, Regina; Kipritidis, John; Gebski, Val; Li, Yifan; Sengupta, Chandrima. Data from: TROG 15.01 SPARK Stereotactic Prostate Adaptive Radiotherapy utilising Kilovoltage intrafraction monitoring (SPARK). 2023. doi: 10.25910/qg5d-6058
25. Rit S, Vila Oliva M, Brousmiche S, Labarbe R, Sarrut D, Sharp G C. The Reconstruction Toolkit (RTK), an open-source cone-beam C T reconstruction toolkit based on the Insight Toolkit (ITK). Journal of Physics: Conference Series. 2014 Mar. 24 2014; 489 (1): 012079. doi: 10.1088/1742-6596/489/1/012079
26. Poulsen P R, Cho B, Keall P J. A Method to Estimate Mean Position, Motion Magnitude, Motion Correlation, and Trajectory of a Tumor From Cone-Beam C T Projections for Image-Guided Radiotherapy. International Journal of Radiation Oncology*Biology*Physics. 2008 Dec. 1/2008;72 (5): 1587-1596. doi: https://doi.org/10.1016/j.ijrobp.2008.07.037
27. Tehrani J N, O'Brien R T, Poulsen P R, Keall P. Real-time estimation of prostate tumor rotation and translation with a kV imaging system based on an iterative closest point algorithm. Physics in Medicine and Biology. 2013 Nov. 15 2013; 58 (23): 8517-8533. doi: 10.1088/0031-9155/58/23/8517
28. Klein S, Staring M, Murphy K, Viergever M A, Pluim JPW. elastix: A Toolbox for Intensity-Based Medical Image Registration. IEEE Transactions on Medical Imaging. 2010; 29 (1): 196-205. doi: 10.1109/TMI.2009.2035616
29. Balter J M, Sandler H M, Lam K, Bree R L, Lichter A S, Ten Haken R K. Measurement of prostate movement over the course of routine radiotherapy using implanted markers. International Journal of Radiation Oncology*Biology*Physics. 1995 Jan. 1/1995;31 (1): 113-118. doi: https://doi.org/10.1016/0360-3016 (94) 00382-U
30. Kupelian P, Willoughby T, Mahadevan A, et al. Multi-institutional clinical experience with the Calypso System in localization and continuous, real-time monitoring of the prostate gland during external radiotherapy. International Journal of Radiation Oncology*Biology*Physics. 2007 Mar. 15/2007; 67 (4): 1088-1098. doi: https://doi.org/10.1016/j.ijrobp.2006.10.026
31. Tyagi N, Hipp E, Cloutier M, et al. Impact of daily soft-tissue image guidance to prostate on pelvic lymph node (PLN) irradiation for prostate patients receiving SBRT. https://doi.org/10.1002/acm2.12665. Journal of Applied Clinical Medical Physics. 2019 Jul. 1 2019; 20 (7): 121-127. doi: https://doi.org/10.1002/acm2.12665
32. Munbodh R, Jaffray D A, Moseley D J, et al. Automated 2D-3D registration of a radiograph and a cone beam C T using line-segment enhancementa). Medical Physics. 2006;33 (5): 1398-1411. doi: https://doi.org/10.1118/1.2192621
33. Munbodh R, Chen Z, Jaffray D A, Moseley D J, Knisely JPS, Duncan J S. Automated 2D-3D registration of portal images and C T data using line-segment enhancement. Medical Physics. 2008;35 (10): 4352-4361. doi: https://doi.org/10.1118/1.2975143
34. Zhang X, Zheng G, Langlotz F, Nolte L P. Assessment of spline-based 2D-3D registration for image-guided spine surgery. Minimally Invasive Therapy & Allied Technologies. 2006 Jan. 1 2006;15 (3): 193-199. doi: 10.1080/13645700600769474
35. Fu D, Kuduvalli G. A fast, accurate, and automatic 2D-3D image registration for image-guided cranial radiosurgery. Medical Physics. 2008;35 (5): 2180-2194. doi: https://doi.org/10.1118/1.2903431
36. Li G, Yang T J, Furtado H, et al. Clinical Assessment of 2D/3D Registration Accuracy in 4 Major Anatomic Sites Using On-Board 2D Kilovoltage Images for 6D Patient Setup. Technology in Cancer Research & Treatment. 2015;14 (3): 305-314. doi: 10.1177/1533034614547454
37. Rossi P J, Schreibmann E, Jani A B, Master V A, Johnstone PAS. Boost first, eliminate systematic error, and individualize CTV to PTV margin when treating lymph nodes in high-risk prostate cancer. Radiotherapy and Oncology. 2009 Mar. 1/2009; 90 (3): 353-358. doi: https://doi.org/10.1016/j.radonc.2008.09.021
38. Björeland U, Jonsson J, Alm M, Beckman L, Nyholm T, Thellenberg-Karlsson C. Inter-fraction movements of the prostate and pelvic lymph nodes during IGRT. Journal of Radiation Oncology. 2018 Dec. 1 2018; 7 (4): 357-366. doi: 10.1007/s13566-018-0366-3
39. Tvilum M, Khalil A A, Møller D S, Hoffmann L, Knap M M. Clinical outcome of image-guided adaptive radiotherapy in the treatment of lung cancer patients. Acta Oncologica. 2015 Oct. 21 2015;54 (9): 1430-1437. doi: 10.3109/0284186X.2015.1062544
40. Kensen C M, Betgen A, Wiersema L, et al. Online Adaptive MRI-Guided Radiotherapy for Primary Tumor and Lymph Node Boosting in Rectal Cancer. Cancers. 2023;15 (4): 1009.
41. Liu F, Tai A, Ahunbay E, Gore E, Johnstone C, Li X A. Management of independent motion between multiple targets in lung cancer radiation therapy. Practical radiation oncology. 2017;7 (1): 26-34.
42. Xia P, Qi P, Hwang A, Kinsey E, Pouliot J, Roach III M. Comparison of three strategies in management of independent movement of the prostate and pelvic lymph nodes. Medical Physics. 2010; 37 (9): 5006-5013. doi: https://doi.org/10.1118/1.3480505
43. Hewson E A, Nguyen D T, Le A, Booth J T, Keall P J, Mejnertsen L. Optimising multi-target multileaf collimator tracking using real-time dose for locally advanced prostate cancer patients. Physics in Medicine & Biology. 2022 Sep. 13 2022;67 (18): 185003. doi: 10.1088/1361-6560/ac8967
44. Hazelaar C, Dahele M, Mostafavi H, van der Weide L, Slotman B J, Verbakel WFAR. Subsecond and Submillimeter Resolution Positional Verification for Stereotactic Irradiation of Spinal Lesions. International Journal of Radiation Oncology*Biology*Physics. 2016 Apr. 1/2016; 94 (5): 1154-1162. doi: https://doi.org/10.1016/j.ijrobp.2016.01.006
45. Hazelaar C, Verbakel WFAR, Mostafavi H, van der Weide L, Slotman B J, Dahele M. First Experience With Markerless Online 3D Spine Position Monitoring During SBRT Delivery Using a Conventional LINAC. International Journal of Radiation Oncology, Biology, Physics. 2018; 101 (5): 1253-1258. doi: 10.1016/j.ijrobp.2018.04.046
46. Roggen T, Bobic M, Givehchi N, Scheib S G. Deep Learning model for markerless tracking in spinal SBRT. Physica Medica. 2020 Jun. 1/2020; 74:66-73. doi: https://doi.org/10.1016/j.ejmp.2020.04.029
47. van der Wielen G J, Mutanga T F, Incrocci L, et al. Deformation of Prostate and Seminal Vesicles Relative to Intraprostatic Fiducial Markers. International Journal of Radiation Oncology*Biology*Physics. 2008 Dec. 1/2008;72 (5): 1604-1611.e3. doi: https://doi.org/10.1016/j.ijrobp.2008.07.023
48. Mak D, Gill S, Paul R, et al. Seminal vesicle interfraction displacement and margins in image guided radiotherapy for prostate cancer. Radiation Oncology. 2012 Aug. 13 2012; 7 (1): 139. doi: 10.1186/1748-717X-7-139
49. Brand V J, Milder MTW, Christianen MEMC, Hoogeman M S, Incrocci L. Seminal vesicle inter- and intra-fraction motion during radiotherapy for prostate cancer: A review. Radiotherapy and Oncology. 2022 Apr. 1/2022; 169:15-24. doi: https://doi.org/10.1016/j.radonc.2022.02.002
50. van Elmpt W, Öllers M, van Herwijnen H, et al. Volume or Position Changes of Primary Lung Tumor During (Chemo-) Radiotherapy Cannot Be Used as a Surrogate for Mediastinal Lymph Node Changes: The Case for Optimal Mediastinal Lymph Node Imaging During Radiotherapy. International Journal of Radiation Oncology*Biology*Physics. 2011 Jan. 1/2011; 79 (1): 89-95. doi: https://doi.org/10.1016/j.ijrobp.2009.10.059
51. Schaake E E, Rossi M M, Buikhuisen W A, et al. Differential motion between mediastinal lymph nodes and primary tumor in radically irradiated lung cancer patients. International Journal of Radiation Oncology* Biology* Physics. 2014; 90 (4): 959-966.
52. Van den Begin R, Engels B, Boussaer M, et al. Motion management during SBRT for oligometastatic cancer: Results of a prospective phase I I trial. Radiotherapy and Oncology. 2016 Jun. 1/2016; 119 (3): 519-524. doi: https://doi.org/10.1016/j.radonc.2016.04.020
53. Menten M J, Fast M F, Nill S, Kamerling C P, McDonald F, Oelfke U. Lung stereotactic body radiotherapy with an M R-linac-Quantifying the impact of the magnetic field and real-time tumor tracking. Radiotherapy and Oncology. 2016 Jun. 1/2016; 119 (3): 461-466. doi: https://doi.org/10.1016/j.radonc.2016.04.019
54. Keall P J, Brighi C, Glide-Hurst C, et al. Integrated MRI-guided radiotherapy-opportunities and challenges. Nature Reviews Clinical Oncology. 2022 Jul. 1 2022;19 (7): 458-470. doi: 10.1038/s41571-022-00631-3

1. A method of monitoring motion of an anatomical feature within a subject's body, the method comprising the steps of:
providing a time series of subject images of a region of the subject's body comprising the anatomical feature;
providing a plurality of reference images of the region of the subject's body, wherein the plurality of reference images comprise a range of projection angles around an imaging axis and perpendicular to the imaging axis through the region of the subject's body;
determining a displacement of the anatomical feature in each subject image using the plurality of reference images; and
monitoring motion of the anatomical feature based on the displacement of the anatomical feature over the time series of subject images.

2. The method according to claim 1, wherein the step of providing a plurality of reference images comprises:
providing a plurality of reference images comprising a range of projection angles around a superior-inferior axis through the region of the subject's body, and for each projection angle around the superior-inferior axis, a range of projection angles around the medial-lateral axis through the region of the subject's body.

3. The method according to claim 1, further comprising the step of:
processing the plurality of reference images to identify a reference image with a projection angle that corresponds most closely to a location of the anatomical feature in each subject image.

4. The method according to claim 3, wherein the step of processing the plurality of reference images comprises:
calculating a correlation between image values of a first subject image and each of the plurality of reference images, and identifying the reference image with the highest correlation.

5. The method according to claim 4, wherein the step of calculating a correlation comprises:
calculating a normalized cross-correlation coefficient between the first subject image and each of the plurality of reference images, and identifying the reference image with the maximum normalized cross-correlation coefficient.

6. The method according to claim 5, wherein the step of determining a displacement comprises:
determining a translation of the anatomical feature along an x-axis (Tₓ) and/or y-axis (T_{y}) based on the respective x and y values that correspond to the maximum normalized cross-correlation coefficient, wherein the x-axis and y-axis are in a plane perpendicular to a z-axis representing an imaging axis through the region of the subject's body.

7. The method according to claim 6, wherein upon determining the translation of the anatomical feature for the first subject image, the method comprises the step of:
determining the translation of the anatomical feature along the x-axis (Tₓ) and/or y-axis (T_{y}) for subsequent subject images based on translations of the plurality of reference images within a limited range from the first subject image.

8. The method according to claim 5, wherein the step of determining a displacement comprises:
determining a rotation of the anatomical feature around an x-axis (Rₓ), y-axis (R_{y}) and/or z-axis (R_{z}) based on respective x, y and z values that correspond to the maximum normalized cross-correlation coefficient, wherein the x-axis and y-axis are in a plane perpendicular to the z-axis representing an imaging axis through the region of the subject's body.

9. The method according to claim 8, wherein upon determining the rotation of the anatomical feature for a first subject image, the method comprises the step of:
determining the rotation of the anatomical feature around the x-axis (Rₓ), y-axis (R_{y}) and/or z-axis (R_{z}) for subsequent subject images based on rotations of the plurality of reference images within a limited range from the first subject image.

10. The method according to claim 1, wherein the step of determining a displacement comprises:
determining a translation of the anatomical feature (T_{z}) along a z-axis representing an imaging axis through the region of the subject's body using a probability model based on a location of the anatomical feature in the plurality of reference images.

11. The method according to claim 10, wherein the probability model is a Gaussian probability density function and the translation of the anatomical feature (T_{z}) is estimated by the Gaussian distribution along the imaging z-axis.

12. The method according to claim 1, wherein the step of determining a displacement comprises:
determining the displacement of the anatomical feature in six degrees of freedom comprising translation and rotation (Tₓ, T_{y}, T_{z}, Rₓ, R_{y}, R_{z}), and
wherein the method further comprises the step of:
transforming the displacement of the anatomical feature (Tₓ, T_{y}, T_{z}, Rₓ, R_{y}, R_{z}) to a frame of reference of the subject's body (T_{LR}, T_{SI}, T_{AP}, R_{LR}, R_{SI}, R_{AP}).

13. The method according to claim 1, wherein one or more steps of the method are performed during delivery of therapy to the subject for monitoring in vivo motion of the anatomical feature within the subject's body.

14. The method according to claim 1, wherein the anatomical feature is the pelvis of the subject.

15. A computer program product for monitoring motion of an anatomical feature within a subject's body, the computer program product comprising a non-transitory computer readable storage medium having program code embodied therewith, the program code executable by one or more processors, to perform a method comprising the steps of:
providing a time series of subject images of a region of the subject's body comprising the anatomical feature;
providing a plurality of reference images of the region of the subject's body, wherein the plurality of reference images comprise a range of projection angles around an imaging axis and perpendicular to the imaging axis through the region of the subject's body;
determining a displacement of the anatomical feature in each subject image using the plurality of reference images; and
monitoring motion of the anatomical feature based on the displacement of the anatomical feature over the time series of subject images.

16. A method of delivering therapy to a subject, the method comprising the steps of:
providing motion data of a first anatomical feature within a region of the subject's body, the motion data being determined according to the method of claim 1;
determining one or more target locations within the region of the subject's body for delivering therapy based on the provided motion data of the first anatomical feature; and
operating a therapy delivery system to deliver therapy to the subject at the one or more target locations.

17. The method according to claim 16, further comprising the steps of:
providing motion data of a second anatomical feature within the region of the subject's body; and
determining one or more target locations within the region of the subject's body for delivering therapy based on the provided motion data of the first anatomical feature and the second anatomical feature.

18. The method according to claim 17, wherein the step of determining one or more target locations comprises:
determining a target location for delivering therapy to each of the first anatomical feature and the second anatomical feature based on the provided motion data of the first anatomical feature and the second anatomical feature; and
operating the therapy delivery system to simultaneously deliver therapy to the subject at both target locations of the first anatomical feature and the second anatomical feature.

19. The method according to claim 17, wherein the first anatomical feature is the pelvis of the subject, and the second anatomical feature is the prostate of the subject.

20. A therapy delivery system comprising:
a therapy system for delivering therapy to a subject;
an imaging system for imaging a region of the subject's body;
one or more processors configured to:
provide motion data of a first anatomical feature within the region of the subject's body, the motion data being determined according to the method of claim 1; and
determine one or more target locations within the region of the subject's body for delivering therapy based on the provided motion data of the first anatomical feature; and
a controller configured to operate the therapy delivery system to deliver therapy to the subject at the one or more target locations.
